SEQUENCE LISTING (45 sequences)

SEQ ID NO: 1 - DNA, 2076 bp, Homo sapiens
cgagcgccagcgcgggaaccgggaaaaggaaaccgtgttgtgtacgtaagattcaggaaacgaaaccaggagccgcgggtgttggcgcaaaggttactcccagacccttttccggctgacttctgagaaggttgcgcagcagctgtgcccgacagtctagaggcgcagaagaggaagccatcgcctggccccggctctctggaccttgtctcgctcgggagcggaaacagcggcagccagagaactgttttaatcatggacaaacaaaactcacagatgaatgcttctcacccggaaacaaacttgccagttgggtatcctcctcagtatccaccgacagcattccaaggacctccaggatatagtggctaccctgggccccaggtcagctacccacccccaccagccggccattcaggtcctggcccagctggctttcctgtcccaaatcagccagtgtataatcagccagtatataatcagccagttggagctgcaggggtaccatggatgccagcgccacagcctccattaaactgtccacctggattagaatatttaagtcagatagatcagatactgattcatcagcaaattgaacttctggaagttttaacaggttttgaaactaataacaaatatgaaattaagaacagctttggacagagggtttactttgcagcggaagatactgattgctgtacccgaaattgctgtgggccatctagaccttttaccttgaggattattgataatatgggtcaagaagtcataactctggagagaccactaagatgtagcagctgttgttgtccctgctgccttcaggagatagaaatccaagctcctcctggtgtaccaataggttatgttattcagacttggcacccatgtctaccaaagtttacaattcaaaatgagaaaagagaggatgtactaaaaataagtggtccatgtgttgtgtgcagctgttgtggagatgttgattttgagattaaatctcttgatgaacagtgtgtggttggcaaaatttccaagcactggactggaattttgagagaggcatttacagacgctgataactttggaatccagttccctttagaccttgatgttaaaatgaaagctgtaatgattggtgcctgtttcctcattgacttcatgttttttgaaagcactggcagccaggaacaaaaatcaggagtgtggtagtggattagtgaaagtctcctcaggaaatctgaagtctgtatattgattgagactatctaaactcatacctgtatgaattaagctgtaaggcctgtagctctggttgtatacttttgcttttcaaattatagtttatcttctgtataactgatttataaaggtttttgtacattttttaatactcattgtcaatttgagaaaaaggacatatgagtttttgcatttattaatgaaacttcctttgaaaaactgctttgaattatgatctctgattcattgtccattttactaccaaatattaactaaggccttattaatttttatataaattatatcttgtcctattaaatctagttacaatttatttcatgcataagagctaatgttattttgcaaatgccatatattcaaaaaagctcaaagataattttctttactattatgttcaaataatattcaatatgcatattatctttaaaaagttaaatgtttttttaatcttcaagaaatcatgctacacttaacttctcctagaagctaatctataccataatattttcatattcacaagatattaaattaccaattttcaaattattgttagtaaagaacaaaatgattctctcccaaagaaagacacattttaaatactccttcactctaaaactctggtattataacttttgaaagttaatatttctacatgaaatgtttagctcttacactctatccttcctagaaaatggtaattgagattactcagatattaattaaatacaatatcatatatatattcacagagtataaacctaaataatgatctattagattcaaatatttgaaataaaaacttgatttttttgt

SEQ ID NO: 2 - protein, 318 aa, Homo sapiens
MDKQNSQMNASHPETNLPVGYPPQYPPTAFQGPPGYSGYPGPQVSYPPPPAGHSGPGPAGFPVPNQPVYNQPVYNQPVGAAGVPWMPAPQPPLNCPPGLEYLSQIDQILIHQQIELLEVLTGFETNNKYEIKNSFGQRVYFAAEDTDCCTRNCCGPSRPFTLRIIDNMGQEVITLERPLRCSSCCCPCCLQEIEIQAPPGVPIGYVIQTWHPCLPKFTIQNEKREDVLKISGPCVVCSCCGDVDFEIKSLDEQCVVGKISKHWTGILREAFTDADNFGIQFPLDLDVKMKAVMIGACFLIDFMFFESTGSQEQKSGVW

SEQ ID NO: 3 - DNA, 1265 bp, Homo sapiens
ggccgaatggagaatgaagccttttcaaattcacctcccagtgaaccaccctcaaaatagaagtgaatgtgaaaccacagatatttcattaaaacatatctgaagataaaaacatacctcaagcttcacagatataggacttctgagctgagaggacctcccggacatattgtctaccctaagcaccaggctggacacactgggaaacaggctgaccacctgggctcccaggccttctacccaggacgtcagcatgactacctagtcccacctgctggcacagctggcattcctgttcaaaatcagccaggtagacctgaaggggtaccatggatgccagcaccaccaccaccattaaactgtccgccaggattggaatacttaagtcagatagatatgatactaattcatcagcaaattgaacttctggaagttctattcagttttgaaagtagtaacatgtatgaaatcaagaacagctttgggcagaggatttattttgcagcagaagatactaatttctgtatccgaaattgctgtgggcggtctagaccttttaccttgaggattactgataatgtgggtcgagaagtcataactctggaaagaccactaagatgtaactgttgttgttgcccctgctgccttcaggagatagaaatccaagctcctcctggtgtaccagtaggttatgttactcagacctggcacccatgtctaacaaagtttacaattaaaaatcagaaaagagaggatgtactaaaaattagtggtccatgtatcgtgtgcagctgtattgcgggtgttgattttgagattacatctcttgatgaacaaattgtggttggcaggatttctaagcactggtctgggtttttaagagaggcatttactgatgctgacaactttggaatccaattccctagagaccttgatgttaaaatgaaagccgtgatgattggtgcctgtttcctcattgactacatgttttttgaaagaactaggtaatgactggaatgtcagagtgtgggagtggattaatgattccggatctttggctaggcaaaatgaaactataactgatctaaacggttccttccttcttctactgtgcaaggaagatgtaaggaaaaactcgcacactatctgtggaactcatttaaattcaaatcctagataaacatttcgcattgaatatttacatggagaaaaatcatcaaacatcaacaattatcaagttaattaataaaaatactaggtattgc

SEQ ID NO: 4 - protein, 224 aa, Homo sapiens
MPAPPPPLNCPPGLEYLSQIDMILIHQQIELLEVLFSFESSNMYEIKNSFGQRIYFAAEDTNFCIRNCCGRSRPFTLRITDNVGREVITLERPLRCNCCCCPCCLQEIEIQAPPGVPVGYVTQTWHPCLTKFTIKNQKREDVLKISGPCIVCSCIAGVDFEITSLDEQIVVGRISKHWSGFLREAFTDADNFGIQFPRDLDVKMKAVMIGACFLIDYMFFERTR

SEQ ID NO: 5 - DNA, 1680 bp, Homo sapiens
cggggccggggtccgagctcgggcccgcctccgcctccgccagctcctgtgagctgccgagtgctaggcacccgggctcttctgggggctccagaactaagccacccagacaccatcatctcgaaaaccccagcccttctcccatggcaggctacttgccccccaaaggctacgccccttcgcccccacctccctaccctgtcacccctgggtacccggagccggcgctacatcctgggcccgggcaggcgccagtgcccgcccaggtacctgccccagctcccggcttcgccctcttcccctcgcctggccccgtggccttggggtctgctgcccccttcttgccactgccaggggtgccttctggcctcgaattcctggtgcagattgatcagattttgattcaccagaaggctgagcgagtggaaacgttcctaggctgggagacctgtaatcggtatgaactgcgctctggggccgggcagcccctgggtcaggcggccgaggagagcaactgctgcgcccgtctgtgctgtggcgcccgccggccgctgcgtgtccgcctggccgaccccggggaccgtgaggtgctgcgtttgctccgcccgctgcactgtggctgcagctgctgcccctgtggcctccaggagatggaagtacaggctccaccaggcaccaccattggccacgtgctacagacctggcatcccttcctccccaagttctccatccaggatgccgatcgccagacagtcttgcgagtggtggggccctgctggacctgtggctgtggcacagacaccaactttgaggtgaagactcgggatgaatcccgcagtgtgggccgcatcagcaagcagtgggggggcctggtccgagaagccctcacagatgcagatgactttggcctacagttcccgctggacctggatgtgagggtgaaggctgtgctgctgggagccacattcctcattgactacatgttctttgagaagcgaggaggcgctgggccctctgccatcaccagttagaggccaccatggtgtgaggagaccatcacctcgaccagaactccagatggtcacctgccctggcccctcctctgggcagcccctttcctccatgtacactgcaggggacagaaggggggccccatccctaccctactccctggccgcctgcccctgtggttcccaaggaggggtatgtatgagagccgctctcctgctacctcccaccactgtcccagcagtccctcggcacacaggcatatcagctttcacactttccccatgcactctctcccacccccttccagggcctctgctccaaaggaggcctctggaacccaggactctggggttttacaagagggctggggtgtggaagggcaagctgcaccaaagacggtggatatagccaccgcccccccgccgctgcctagcatctgcttggccaattagttcagcctcagaccatggcactttgagggggtctctacctccccatcaacagctgcagggggaccccagtgccaacttcctctcccactagggccctgccttcagctggtgcttgctgcgattcctgtgccttatgtaactgcccttccttcccttgccctaggaaaaaggctgcatctttatatgttacattcatataaactttgtaactttttgg

SEQ ID NO: 6 - protein, 295 aa, Homo sapiens
MAGYLPPKGYAPSPPPPYPVTPGYPEPALHPGPGQAPVPAQVPAPAPGFALFPSPGPVALGSAAPFLPLPGVPSGLEFLVQIDQILIHQKAERVETFLGWETCNRYELRSGAGQPLGQAAEESNCCARLCCGARRPLRVRLADPGDREVLRLLRPLHCGCSCCPCGLQEMEVQAPPGTTIGHVLQTWHPFLPKFSIQDADRQTVLRVVGPCWTCGCGTDTNFEVKTRDESRSVGRISKQWGGLVREALTDADDFGLQFPLDLDVRVKAVLLGATFLIDYMFFEKRGGAGPSAITS

SEQ ID NO: 7 - DNA, 3206 bp, Homo sapiens
gtttataacttgaaaaatcctctccgtctcccttccctgcctcctttcctttccctttcctctgccagtacaactagacccggcgtctggcgtccccggtgcccagcattctgcggggcaggcggattaattggaattcttcaaaatgtcaggtgtggtacccacagcccctgaacagcctgcaggtgaaatggaaaatcaaacaaaaccaccagatccaaggcctgatgctcctcctgaatacagttctcattttttaccaggaccccctggaacagctgtccctccacctactggctacccaggaggcttgcctatgggatactacagtccacagcaacccagtaccttccctttgtaccagccagttggtggtatccatcctgtccggtatcagcctggcaaatatcctatgccaaatcagtctgttccaataacatggatgccagggccaactcctatggcaaactgccctcctggtctggaatacttagttcagttggacaacatacatgttcttcagcattttgagcctctggaaatgatgacatgttttgaaactaataatagatatgatattaaaaacaactcagaccagatggtttacgttgtaaccgaagacacagatgactttaccaggaatgcctatcggacactaaggcccttcgtcctccgggtcactgattgtatgggccgagaaatcatgacaatgcagagacccttcagatgcacctgctgttgcttctgttgcccctctgccagacaagagctggaggtgcagtgtcctcctggtgtcaccattggctttgttgcggaacattggaacctgtgcagggcggtgtacagcatccaaaatgagaagaaagaaaatgtgatgagagttcgtgggccatgctcaacctatggctgtggttcagattctgtttttgaggtcaaatcccttgatggcatatccaacatcggcagtattatccggaagtggaatggtttgttatcagcaatggcagatgctgaccattttgacattcacttcccactagacctggatgtgaagatgaaagccatgatttttggagcttgcttcctcattgacttcatgtattttgaaagatctccaccacaacgttcaagatagagagacacagcaagccatcaactatggttaattttgaaaaatggaaaagttggattgggcttacagtcagcactcagttatttgcaagtgtatttctttgctttgtagagtatttttattgggtgttaactttgacagctgagagtgggcttgcaagaacacaatctaaaagtgtgtttcaattgagtatctctctagtagaataggagttcatcctgaaaagctgtgactcattaacccagtaaacatatacaaagtaagcttaaaacactataaacatgagataagggaaaatgaatccagagttctcatattaataggtagtgaaacaataaggctttttagagcagactttgttggcataaaataacctggcttctatccctaaccctttcctacctttcctctccgtcaacatgtcctcatactgaagacaaacttgtttcaatgatagtcttcatttttaaaaacaaaaaggcaggcagacagaaataatgatgttttcttgcactaagaaggtactacttgtacacatatatcaaaacctcattctgcaaagtttttgaaggtttcaatgggaaatttgattttattacaaaataaaacattttttaatgttaaagtttatatattccatgcttgttttctcattcactggcatggatgatcaggagctgcctatatatgaaggcagaatcagactatcaggaaaggagctggccagggccacagccagtcaagatctctgagcaacttagagacattggtgtcattatatgaagcttgcatttaatacatttatacataatacatttgtacatttaattcataacgtctcttggtcacagatgccttatatataaaataagttgccagatctctaagattgcctagtacacctttgtatctcatttgatgtgatacccagaagagatcattgttttttgtttttgtttttgtttttttcaagaagatccttcgtgatcaccatgctgttctcatggtaagaactggagttatgtttttaaatttgaaaatatgacattttatgtagcactatataaaaagtgaaagcgacaaattccaccgctgcttaatactgctttgcttctttttattgacatgatagatacatatgtatctacacagagtaataataataaaacacagtaaacattctatttctctatggtctacagcatgccagtaaataatatgtaggaccaataataaattatcaattacacatttttgtgttaactaattaaaagcatagtgtataagtgagtacactctaattaacttgcttctgttgcactttagttttctacctgcatatggactgcatttttttttttaacacagtcagtatgtagaatgggatgtattcttctgctgctgcttattaaataaagaaagcctgagtgttcttagatggggttattctgagatgagggtcttagcctacagttctttttgaaatgaaaggtgctttgttttttaattatattcatcttttcagggtaaatttgtttttctgagtttctcgtaatgctcatttttacatgctgctactagcttttttttttaaaaaaagtaaaagttgctgctttctaaaatattaattgccttatatttgaaagtgccattgcaatcgtaagtagactatgtatttcctataatgatgtctgatatttaaataggaaatcagacaaacaatattcagaaagtttaagcatataaactttttatttttaacttgcctagatccctgtattccaaaacctgctgcatcataataaatatatctatatatatttagcataagacgtgatatttttaatttcttttttaaaaaattatatttgtctcttagagttaaaattttctttatataatattgtcatatgtcatagttttaatacaattcacatgatttctatgtttcttaatgatattttgttgtgtaaaattgatcggattgattaaaaaacaaattctctggaatttgtgcgttcatgctttttcgtattctttatggcttttaaataaatatacaatggttaatagt

SEQ ID NO: 8 - protein, 329 aa, Homo sapiens
MSGVVPTAPEQPAGEMENQTKPPDPRPDAPPEYSSHFLPGPPGTAVPPPTGYPGGLPMGYYSPQQPSTFPLYQPVGGIHPVRYQPGKYPMPNQSVPITWMPGPTPMANCPPGLEYLVQLDNIHVLQHFEPLEMMTCFETNNRYDIKNNSDQMVYVVTEDTDDFTRNAYRTLRPFVLRVTDCMGREIMTMQRPFRCTCCCFCCPSARQELEVQCPPGVTIGFVAEHWNLCRAVYSIQNEKKENVMRVRGPCSTYGCGSDSVFEVKSLDGISNIGSIIRKWNGLLSAMADADHFDIHFPLDLDVKMKAMIFGACFLIDFMYFERSPPQRSR

SEQ ID NO: 9 - DNA, 1502 bp, Mus musculus
gggcagagctcgcgtcattcagagtgagtcccctctgcgagggaaagccgggctactgcagcgccaccagcaagggattgggactcacggacttcagaacagaaggagcctcagaaactgtttaaatcatggaaaaccacagcaagcaaactgaggctccccacccgggaacatatatgccagctgggtatccccctccgtatccaccagcagctttccaaggaccttcagaccatgctgcttaccccataccccaggctggctaccaagggcctccgggcccctatccagggccccaacctggctacccagtcccaccaggaggttatgcaggtggtggccctagtggctttcctgtccaaaatcagccagcatataatcatccaggtgggcctggggggaccccatggatgccagccccccctcctccactgaactgtccaccggggctggaatacttagctcagattgatcagcttctggttcatcagcaaattgagcttctggaagtcttaacaggctttgaaacaaataacaaatatgaaatcaagaacagcctcgggcagagagtttactttgcagtggaagatactgactgctgtacccgaaactgctgtggggcgtctagacctttcaccttgaggatcctggataatctgggccgagaagtcatgactctggagagacctctgagatgcagtagctgctgcttcccctgctgcctccaggagatagaaatccaggctcctcctggggtaccagtaggttatgtgactcagacctggcacccatgtctgcccaagttcactctccaaaatgagaagaagcaggatgtcctgaaagtagttggtccgtgtgttgtgtgtagctgctgttccgacattgactttgagctcaaatctctagatgaagaatcagtagttggcaaaatttctaagcagtggtctggttttgtgagagaggccttcacggatgcagacaattttgggatccagttcccgctagacctggatgtgaagatgaaagctgtgatgcttggtgcttgtttcctcatagatttcatgttttttgaaagaactggaaacgaggagcaaagatcaggagcatggcagtaactccctgagagttcttgaggtttaaggacgacaactttatggaccctgaatggaaactgaggaatcacaaggcacacaccgtggcttcttttcctttactgaaataactttctatcaactcacctgtgatgcctgggtgccctgttgtacaattatgctcccaaattagagtttattttttagaattctgtcatgtatttgtttttatacattcttaaggttttcactgtgaatttgggaaaacagttatgtgaatttatatacatagaaatgatcttctctatgaaaacatactttgactttgtctttcgtttcccatttttgtggaaacgtaaatgctattgtaatttaatataaaattacacattaaatataattatgatttac

SEQ ID NO: 10 - protein, 328 aa, Mus musculus
MENHSKQTEAPHPGTYMPAGYPPPYPPAAFQGPSDHAAYPIPQAGYQGPPGPYPGPQPGYPVPPGGYAGGGPSGFPVQNQPAYNHPGGPGGTPWMPAPPPPLNCPPGLEYLAQIDQLLVHQQIELLEVLTGFETNNKYEIKNSLGQRVYFAVEDTDCCTRNCCGASRPFTLRILDNLGREVMTLERPLRCSSCCFPCCLQEIEIQAPPGVPVGYVTQTWHPCLPKFTLQNEKKQDVLKVVGPCVVCSCCSDIDFELKSLDEESVVGKISKQWSGFVREAFTDADNFGIQFPLDLDVKMKAVMLGACFLIDFMFFERTGNEEQRSGAWQ

SEQ ID NO: 11 - DNA, 1622 bp, Mus musculus
tctaaagactcaggaaacaaaacctaaattgcctcaaagttcaggtgctttttctccctgactttagtctagtggagtagtgcagcacctatgcctttctgagaggagtctggagagctgagtcgctgctggtgctaggattctaggaattcgcctcacttggagctgcatgagaaaagaaaggcttgcaaatggaggctcctcgctcaggaacatacttgccagctgggtatgcccctcagtatcctccagcagcagtccaaggacctccagagcatactggacgccccacattccagactaactaccaagttccccagtctggttatccaggacctcaggctagctacacagtctcaacatctggacatgaaggttatgctgctacacggcttcctattcaaaataatcagactatagtccttgcaaacactcagtggatgccagcaccaccacctattctgaactgcccacctgggctagaatacttaaatcagatagatcagcttctgattcatcagcaagttgaacttctagaagtcttaacaggctttgaaacaaataacaaatttgaaatcaagaacagcctcgggcagatggtttatgttgcagtggaagatactgactgctgtactcgaaattgctgtgaagcgtctagacctttcaccttaagaatcctggatcatctgggccaagaagtcatgactctggagcgacctctgagatgcagtagctgctgcttcccctgctgcctccaggagatagaaatccaggctcctccgggggtgccaataggttatgtgactcagacctggcacccatgtctgccaaagctcactcttcagaacgacaagagggagaatgttctaaaagtagttggtccatgtgttgcatgcacctgctgttcagatattgactttgagatcaagtctcttgatgaagtgactagaattggtaagatcaccaagcagtggtctggttgtgtgaaagaggccttcacggattcggataactttgggatccaattcccgctagacctggaggtgaagatgaaagctgtgacgcttggtgcttgcttcctcatagattacatgttttttgaaggctgtgagtaggaacagaaatccgacctgcagtaggaatcaatgaaagaggacagagaagatctgaagtctacacaaggagatcatatgattgagagacctggggctttttgatttcttcattgaaatttctcagaatcaagctgttatacatgaagcatagtatgtaacattttggttttcaaatggtagtttatcttttacattattggaatagacctggataattatctttatacacttctaaaaatatgcaccaaattcaagttaaaaaaaaaaagacgaagagaagtgtatgttttaaaataaaacattttatggaaaagtaagttaaatcataatctgggatttatttttcatcttttgttcaatttaaaccttgttagtgctgattttattataaaattgtactttactatcaaacctagttagtttatttcttacagaaatcctcctattattttgaaattacatatttttgaaagctttttaaaagatactattgcctgggaaattcta

SEQ ID NO: 12 - protein, 307 aa, Mus musculus
MEAPRSGTYLPAGYAPQYPPAAVQGPPEHTGRPTFQTNYQVPQSGYPGPQASYTVSTSGHEGYAATRLPIQNNQTIVLANTQWMPAPPPILNCPPGLEYLNQIDQLLIHQQVELLEVLTGFETNNKFEIKNSLGQMVYVAVEDTDCCTRNCCEASRPFTLRILDHLGQEVMTLERPLRCSSCCFPCCLQEIEIQAPPGVPIGYVTQTWHPCLPKLTLQNDKRENVLKVVGPCVACTCCSDIDFEIKSLDEVTRIGKITKQWSGCVKEAFTDSDNFGIQFPLDLEVKMKAVTLGACFLIDYMFFEGCE

SEQ ID NO: 13 - DNA, 1708 bp, Mus musculus
ccccgagtcttaggtgccgccctagagaccctgggcccgtactgggcgcagctacctcttcgcctctgcctgtccgtctttgtttctgtgtctgtctagctgttcccgagcttgtcccactccagaactaagtctcccctacgccaaaagcccaagactcccctcctgattcccatggcaggctacttgccccccaaaggctatgccccttcacccccacctccctaccccgtgccatctgggtatccagagccggtggctctgcatcctggaccgggacaagctccagtgcccacccaggtgcctgcccctgctcccggcttcgctctcttcccctcgccaggcccagtggctccagggcctcctgctcctttcgtgccattgccaggggtgcctcctggcctcgaattcctagtgcagattgatcaaatcttgattcatcagaaggctgaacgagtggaaacgttcctaggctgggagacctgtaatatgtatgaacttcgctccggaaccggacagcaactgggtcaggcagctgaagagagcaactgttgtgcccgcctgtgctgtggtgcccgccgaccatttcgaatccgcctagcggaccctggggaccgcgaggtgctccggctcctccgcccacttcattgtggctgcagctgctgcccctgtggtcttcaggagatggaagtccaggctccacctggcaccaccattggccatgtgctacagacctggcatcccttccttcctaagttttccatcctggatgctgatcgccaacctgttctacgagttgtagggccttgctggacttgtggctgtggtacagacaccaactttgaggtgaagactaaggatgaatcgcgaagtgtgggccgcatcagcaagcagtggggagggctgctccgagaagccctcacagatgccgatgactttggcctccaattcccagtcgatctagatgtgaaagtgaaggccgtactgctgggagccacgttcctcatcgattatatgttcttcgagaagagaggaggcgcaggaccctctgccatcaccagttagaagccacctcaggatgaggagacccatctccttgaccagaatttaagatggtcagctgccctggacgttccctcctgaagcaaccctttccttgatatacactgcggcggaccgacgagggtggccgagtggttgggagccgttgtgtcccatcccttccctgcttctcctgtggccctgcagaagagcatgtatgagacctgttcctccttctgttcaccatctccaggcagtgcctgtgcacacattagcttttaaacttccttgcacactccttccagccttcctctggggcctctgcataggcaggggcatctggaatcctggactcaagttttaccccagggcttgtgggtaaaaggcaagcagtaccaaagatggcagacaccacccttcccttatggcactttagccaattagtttagcttccgattgtggcactctgaggggatccttgcctcctcactaatagctgtagcggttgggccccagtgccaactccctaagcccctgggccctgcgggtgctttctgcagcttcctgtgccttatttaaccgttaaccccttccttcccctactgtaggaaggaggctgtgtctttgtatgttgtactcatataaactttgaaactttttaaacagt

SEQ ID NO: 14 - protein, 296 aa, Mus musculus
MAGYLPPKGYAPSPPPPYPVPSGYPEPVALHPGPGQAPVPTQVPAPAPGFALFPSPGPVAPGPPAPFVPLPGVPPGLEFLVQIDQILIHQKAERVETFLGWETCNMYELRSGTGQQLGQAAEESNCCARLCCGARRPFRIRLADPGDREVLRLLRPLHCGCSCCPCGLQEMEVQAPPGTTIGHVLQTWHPFLPKFSILDADRQPVLRVVGPCWTCGCGTDTNFEVKTKDESRSVGRISKQWGGLLREALTDADDFGLQFPVDLDVKVKAVLLGATFLIDYMFFEKRGGAGPSAITS

SEQ ID NO: 15 - DNA, 2162 bp, Mus musculus
  misc_feature (1)..(2162): n is any nucleotide
cagatgactttaccaggaatgcctatcggaacctacgaccctttgtgctccgggtcactgactgcctgggccgagagatcatgaccatgcagaggcctttccgatgcacctgctgttgcttctgctgcccctgtgcaagacaagagctggaagtgcaatgtcctcctggggtcaccattggctttgttgcagaacactggaacttgtgcagagcctcttatagcatccagaatgagaagaaagagagtatgatgagagtgaggggtccgtgtgcaacctacggctgtggctccgattctgtttttgagatcaactctctagatggcgtgtctaacatcggcagtattataaggaagtggaatggctttttatcaacgatggtaaatgctgaccactttgagattcgcttccctttggccctggatgtgaagatgaaagcaatgattttcggctcttgcttcctcattgacttcatgtactttgaacgacctcctccgcgacgtatgtcaagatagaggatcaaacaaatcatcaattgaaaaaaaatgagtaatttgcattcagcttatgtatagctttctgttattagcacatatatttctctggcttgacatgtgtctcagttgagtatttagcttcttgaggcactaggttattctgaaatctctactccattagccaaataaacatatacagatacaagcattacagctataggcatgtgagctatgggaaagtgaaacccttggttctcaacttgacaggtcacaacacatcttctgtncctgtccctttcttcttcattaggaaaaacttgattccaggtgagaggactgctactcttagtcttcatctttaggagccaaaaagtgcacagaaataatgtgctttaatgtgctcagaaaatgttttgtgtgtgtgtgtgtgtctgtgtgcaccagaacatcattctgcaacatgcatacaagttcctaaagttctcagtggggaattttattttattacaaaataaaatgtattctctttataggcagtttatattttctatgcatatttcttcataccctcaactgggcaatgtggagctacctgtatatgaaggtaggatgagggtaccagggaaagttgatcagagctacagccaaataagttcttggcatcttacacatgttgtgtgtttgcttactcagcaaaattagtgtggactctcgtatttacacttgccttttatctaactgtttatactgatgtctggaattacctattgtatttttgtatctcacttgctatttttttttttttcgaaatccatctaatgaccaggatgtccttgtgaccaacagtttattaaagctgaatatgtaaatcctgtcattgctttaatactggtgacgctttgcgtggtaaacaactcattatatgcaatcagtaagaagacacagtaaatttgttgcttccatattttctccagattatcaatgaggactatggaagaccagcagacttgtactaaggacagtgtatgtatgaatcactgatttaattaatgagtttcatgcctttgctgtcgacctacacatagatgctgtatgggttttttttttaatggatatattctcttanagtttatttaaaaaggattaagtgctcttgggttgaattatcctgagagaagagatgtagccaacaggcctttttgaaatagcaggngcttcagctttaattatactaatgtttttatgagaaagattgttctcctcagttatctacttgctgctgttgagtttttatttaaaaaaaatcatgagttcttgactaaaaactaccttaaatgttaaaatgtacctctaattttaaccagacatacatgatttctgtatgacatcttgtattttgcatataaaccagataaacagaattctgagagctcacgtataagcctgtcctttgcccacccacaccccattctagcactttgcatcacgttgatagtgaaagtcagaatgcttcatatttctgtgctccatacttcagtgttcatgatgctaatacttctctaacagcagaattctttaacataagtcatgcaatttctgtaacgcagtagaatttcactgtatcaagacagcctacttaactaaaaaataaacaacttgttttct

SEQ ID NO: 16 - protein, 170 aa, Mus musculus
DDFTRNAYRNLRPFVLRVTDCLGREIMTMQRPFRCTCCCFCCPCARQELEVQCPPGVTIGFVAEHWNLCRASYSIQNEKKESMMRVRGPCATYGCGSDSVFEINSLDGVSNIGSIIRKWNGFLSTMVNADHFEIRFPLALDVKMKAMIFGSCFLIDFMYFERPPPRRMSR

SEQ ID NO: 17 - DNA, 210 bp, Homo sapiens
agtttcctctccttaaccaccggacaaacgtctctggagtctctccaatgagcaagaaagcaagtcgggggtaggggaggggcctcacaccagggggtgggcgcagtccctcctccagctccttcaccctccagtagtctcgtgggtccccgagcgccagcgcgggaaccgggaaaaggaaaccgtgttgtgtacgtaagattcaggaaa

SEQ ID NO: 18 - DNA, 25 bp, Artificial sequence
  Forward primer for PCR
cctggtgcttagggtagacaatatg

SEQ ID NO: 19 - DNA, 26 bp, Artificial sequence
  Reverse primer for PCR
ctgacgtcctgggtagaaggcctggg

SEQ ID NO: 20 - DNA, 23 bp, Artificial sequence
  Forward primer for PCR
tgtgaggagaccatcacctcgac

SEQ ID NO: 21 - DNA, 22 bp, Artificial sequence
  Reverse primer for PCR
aaagctgatatgcctgtgtgcc

SEQ ID NO: 22 - DNA, 24 bp, Artificial sequence
  T7 promoter sequence contained in reverse primer
aatttaatacgactcactataggg

SEQ ID NO: 23 - DNA, 14 bp, Artificial sequence
  HuPLSCR1 GC box
taggggaggggcct

SEQ ID NO: 24 - DNA, 14 bp, Artificial sequence
  HuPLSCR1 GC box
aggaggtgggcgca

SEQ ID NO: 25 - DNA, 11 bp, Artificial sequence
  HuPLSCR1 CCAAT box
tctctccaatg

SEQ ID NO: 26 - DNA, 16 bp, Artificial sequence
  Human Scramblase Splice donor site 1
agccagaggtgcgcgg

SEQ ID NO: 27 - DNA, 16 bp, Artificial sequence
  Human Scramblase Splice acceptor site 1
tttttcagaactgttt

SEQ ID NO: 28 - DNA, 16 bp, Artificial sequence
  Human Scramblase Splice donor site 2
caaacaaagtaagtaa

SEQ ID NO: 29 - DNA, 16 bp, Artificial sequence
  Human Scramblase Splice acceptor site 2
aattgcagactcacag

SEQ ID NO: 30 - DNA, 16 bp, Artificial sequence
  Human Scramblase Splice donor site 3
attccaaggtaaagca

SEQ ID NO: 31 - DNA, 16 bp, Artificial sequence
  Human Scramblase Splice acceptor site 3
tatttcaggacctcca

SEQ ID NO: 32 - DNA, 16 bp, Artificial sequence
  Human Scramblase Splice donor site 4
taagtcaggtaatttc

SEQ ID NO: 33 - DNA, 16 bp, Artificial sequence
  Human Scramblase Splice acceptor site 4
tgctatagatagatca

SEQ ID NO: 34 - DNA, 16 bp, Artificial sequence
  Human Scramblase Splice donor site 5
tctggaaggtatgtat

SEQ ID NO: 35 - DNA, 16 bp, Artificial sequence
  Human Scramblase Splice acceptor site 5
gtttttagttttaaca

SEQ ID NO: 36 - DNA, 16 bp, Artificial sequence
  Human Scramblase Splice donor site 6
ttcaggaggtctgtga

SEQ ID NO: 37 - DNA, 16 bp, Artificial sequence
  Human Scramblase Splice acceptor site 6
ctttgtagatagaaat

SEQ ID NO: 38 - DNA, 16 bp, Artificial sequence
  Human Scramblase Splice donor site 7
attttgaggtaagaga

SEQ ID NO: 39 - DNA, 16 bp, Artificial sequence
  Human Scramblase Splice acceptor site 7
caatttagattaaatc

SEQ ID NO: 40 - DNA, 16 bp, Artificial sequence
  Human Scramblase Splice donor site 8
tcctcattgtaagtct

SEQ ID NO: 41 - DNA, 16 bp, Artificial sequence
  Human Scramblase Splice acceptor site 8
ttatctaggacttcat

SEQ ID NO: 42 - DNA, 13 bp, Homo sapiens
gaaaagagaatcc

SEQ ID NO: 43 - DNA, 13 bp, Homo sapiens
acaaaaagaaagc

SEQ ID NO: 44 - DNA, 13 bp, Homo sapiens
aaaaacagaaacc

SEQ ID NO: 45 - DNA, 13 bp, Homo sapiens
ggaaaaggaaacc

(19) United States
(12) Patent Application Publication
(10) Pub. No.: US 2002/0137905 A1
(43) Pub. Date: Sep. 26, 2002

(54) Phospholipid Scramblases and methods of use thereof

(76) Inventors: Peter J. Sims, Del Mar, CA (US); Therese Wiedmer, Del Mar, CA (US); Robert H. Silverman, Beachwood, OH (US)

Correspondence Address: Lisa A. Haile, Ph.D., Gray Cary Ware & Freidenrich LLP, 4365 Executive Drive, Suite 1600, San Diego, CA, 92121-2189, US

(21) Appl. No.: 09/823,847

(22) Filed: Mar. 30, 2001

Related U.S. Application Data
(60) Provisional application No. 60/193,939, filed on Mar. 31, 2000.

(51) Int. Cl. C07H 21/02; C07H 21/04; A61K 38/46; C12N 9/16

(52) U.S. Cl. 536/23.1; 435/196; 424/94.6

(57) ABSTRACT

The present invention is based on a family of membrane proteins, Phospholipid Scramblases (PLSCR), that mediate accelerated trans-bilayer movement of plasma membrane phospholipids in response to elevated cytoplasmic calcium. At least one Phospholipid Scramblase gene is highly inducible by interferon. Interferon-induced expression of Phospholipid Scramblase 1 (and/or related genes) alters the physical and functional properties of the cell surface so as to (1) inhibit tumor cell proliferation and survival; (2) inhibit maturation and release of membrane-enveloped viruses; and/or (3) promote clearance of virus-infected cells and cancer cells through the reticuloendothelial system. The present invention provides Phospholipid Scramblase polypeptides, polynucleotide sequences that encode Phospholipid Scramblase polypeptides, and antibodies that are immunoreactive with the polypeptides. The finding that human Phospholipid Scramblase 1 polypeptides are induced by interferons, indicates a role for the Scramblase polypeptides in treating and preventing cancer and viral infection.

### CROSS-RELATED APPLICATIONS

This application claims priority under 35 U.S.C. § 119(e)(1) to U.S. provisional application Ser. No. 60/193,939 filed Mar. 31, 2000, which is incorporated by reference herein in its entirety.

### FIELD OF THE INVENTION

The present invention relates generally to cellular membrane proteins that mediate trans-bilayer movement of membrane phospholipids and more specifically to a group of Phospholipid Scramblases and methods of use for preventing and treating viral infection and cancer.

### BACKGROUND OF THE INVENTION

Cancer arises from a loss of normal growth control. In normal tissues, the rates of new cell growth and old cell death are kept in balance. In cancer, this balance is disrupted. This disruption can result from uncontrolled cell growth or loss of a cell's ability to undergo “apoptosis.” Apoptosis, or “cell suicide,” is the mechanism by which old or damaged cells normally self-destruct.

Cancer can originate almost anywhere in the body. Carcinomas, the most common types of cancer, arise from the cells that cover external and internal body surfaces. Lung, breast, and colon are the most frequent cancers of this type in the United States. Sarcomas are cancers arising from cells found in the supporting tissues of the body such as bone, cartilage, fat, connective tissue, and muscle. Lymphomas are cancers that arise in the lymph nodes and tissues of the body's immune system. Leukemias are cancers of the immature blood cells that grow in the bone marrow and tend to accumulate in large numbers in the bloodstream.

The increase in the number of dividing cells creates a growing mass of tissue called a “tumor” or “neoplasm.” If the rate of cell division is relatively rapid, and no “suicide” signals are in place to trigger cell death, the tumor will grow quickly in size; if the cells divide more slowly, tumor growth will be slower. But regardless of the growth rate, tumors ultimately increase in size because new cells are being produced in greater numbers than needed. As more and more of these dividing cells accumulate, the normal organization of the tissue gradually becomes disrupted.

Detecting cancer early can affect the outcome of the disease for some cancers. When cancer is found, one can determine what type it is and how fast it is growing. It can also be determined whether cancer cells have invaded nearby healthy tissue or metastasized to other parts of the body. In some cases, finding cancer early may decrease a person's risk of dying from the cancer. For this reason, improving methods for early detection is currently a high priority for health care workers.

Another health concern is viral infection, caused by viruses. Viruses, the smallest human pathogens, range in size from 20 to 300 nm and consist of RNA or DNA contained in a protein shell. Some viruses are enveloped in a lipid membrane. Viruses are incapable of independent metabolism or reproduction and thus are obligated to use living cells for replication. After invading cells, these microorganisms divert their biosynthetic and metabolic capacities to the synthesis of viral-encoded nucleic acids and proteins.

Viruses can cause disease by killing infected cells. Viruses also produce disease by promoting the release of chemical mediators that incite inflammatory or immunological responses. Some viruses produce disease by causing cells to proliferate and form tumors.

Various signalling compounds are involved in the cellular and molecular response to cancer and viral infection. Cytokines are well known in the art and include, but are not limited to the tumor necrosis factors (TNFs), colony stimulating factors (CSFs), interferons (INFs), interleukins, transforming growth factors (TGFs), oncostatin M (OSM), leukemia inhibiting factor (LIF), platelet activating factor (PAF) and other soluble immunoregulatory peptides that mediate host defense responses, cell regulation and cell differentiation (see, for example, Kuby, Immunology 2d ed. (W. H. Freeman and Co. 1994); see Chapter 13.

Thus, there is a continuing need in the art for methods and compounds that can specifically inhibit or prevent cancer and viral infection.

### SUMMARY OF THE INVENTION

The present invention is based on the discovery of a family of membrane proteins, Phospholipid Scramblases (PLSCR), that are thought to mediate accelerated trans-bilayer movement of plasma membrane phospholipids in response to elevated cytoplasmic calcium. Phospholipid Scramblases are involved in the de novo movement of phosphatidylserine and other aminophospholipids to the plasma membrane outer leaflet following cellular injury and it may contribute to cell surface phosphatidylserine exposure during early stages of programmed cell death. The cell surface exposure of these aminophospholipids is known to promote activation of plasma complement and coagulation proteases and to promote clearance of cells by the reticuloendothelial system.

At least one Phospholipid Scramblase gene is highly inducible by interferon. Interferons (IFNs) are pleiotropic cytokines with antiviral, immunoregulatory and antiproliferative activities that are used clinically against malignancies, viral infections and multiple sclerosis. Interferon-induced expression of Phospholipid Scramblase 1 (and/or related genes) alters the physical and functional properties of the cell surface so as to (1) inhibit tumor cell proliferation and survival; (2) inhibit maturation and release of membrane-enveloped viruses; and/or (3) promote clearance of virus-infected cells and cancer cells through the reticuloendothelial system.

The present invention provides Phospholipid Scramblase polypeptides, polynucleotide sequences that encode Phospholipid Scramblase polypeptides, and antibodies that are immunoreactive with the polypeptides. The finding that human Phospholipid Scramblase 1 polypeptides are induced by interferons, indicates a role for the Scramblase polypeptides in treating and preventing cancer and viral infection.

In a first embodiment, there is provided isolated Phospholipid Scramblase polynucleotides (a) having the nucleotide sequences as set forth in SEQ ID NO:3, SEQ ID NO:5, SEQ ID NO:7, SEQ ID NO:9. SEQ ID NO:13, and SEQ ID NO:15; (b) polynucleotides of (a), wherein T can be U; (c) polynucleotides complementary to (a); and (d) fragments of (a), (b), or (c), having at least 15 base pairs and that hybridizes to DNA that encodes the Phospholipid Scramblase polypeptides as set forth in SEQ ID NO:4, SEQ ID NO:6. SEQ ID NO:8, SEQ ID NO:10, SEQ ID NO:14 and SEQ ID NO:16.

In another embodiment there is provided a substantially purified Phospholipid Scramblase polypeptide that has the sequence encoded by a polynucleotide set forth as SEQ ID NO:3, SEQ ID NO:5, SEQ ID NO:7, SEQ ID NO:13, or SEQ ID NO:15. Also provided is a substantially purified Phospholipid Scramblase polypeptide having the amino acid sequence set forth in SEQ ID NO:4, SEQ ID NO:6, SEQ ID NO:8, SEQ ID NO:14, or SEQ ID NO:16.

In yet another embodiment there is provided an antibody that binds to a Phospholipid Scramblase polypeptide or immunoreactive fragments of the polypeptide. Such antibodies are useful for detecting Phospholipid Scramblase in patient samples, for example. Antibodies of the invention include antibodies that distinguish PLSCR from other proteins and antibodies that distinguish among the different PLSCR family members.

In still another embodiment there is provided an expression vector containing a polynucleotide sequence of the invention. The invention also provides a host cell containing the expression vector. In another embodiment, the invention provides a method for producing a Phospholipid Scramblase polypeptide of the invention or biologically active or immunogenic fragments thereof. An exemplary Phospholipid Scramblase polypeptide, having the amino acid sequence of SEQ ID NO:4, is produced by culturing a host cell containing an expression vector and recovering the polypeptide from the host cell culture.

In another embodiment, the invention provides an isolated nucleic acid sequence containing a non-coding regulatory sequence isolated upstream from a Phospholipid Scramblase gene. Preferably the regulatory sequence contains at least one restriction site for cloning a heterologous nucleic acid sequence of interest. The isolated nucleic acid sequence can be operably linked to a heterologous nucleic acid sequence to form a DNA construct, and the heterologous sequence can be a selectable marker sequence, or a reporter gene, for example.

In yet another embodiment, the invention provides a method for identifying a compound that modulates expression of a Phospholipid Scramblase polypeptide. The method includes incubating the compound with a cell containing a DNA construct containing a PLSCR regulatory sequence and encoding a heterologous polypeptide under conditions sufficient to permit the compound to interact with the regulatory sequence of the construct; and detecting expression of the heterologous gene in the presence and absence of the compound to identify a compound that modulates Phospholipid Scramblase polypeptide expression. The cellular response may be a decrease or an increase in calcium mobilization for example. The modulation may be an inhibition of or stimulation of Phospholipid Scramblase expression.

Also included is a transgenic non-human animal whose genome comprises a disruption of a Phospholipid Scramblase polypeptide gene, wherein the disruption results in the animal exhibiting a higher susceptibility to viral infection or cancer as compared to a wild-type animal not having the disruption and a method for making the same.

The invention includes a method of inhibiting or preventing viral infection in a subject. The method includes contacting viral-infected cells or uninfected cells with a Phospholipid Scramblase polypeptide or fragments thereof that contain an amino acid sequence PpxY thereby inhibiting or preventing viral infection. The method can also include co-administering interferon, (e.g., α, β or γ) either prior to, simultaneously with or immediately following PLSCR administration.

In yet another embodiment, the invention provides a method for identifying a compound that modulates Phospholipid Scramblase polypeptide activity. The method includes incubating a test compound and a cell expressing Phospholipid Scramblase polypeptide under conditions sufficient to permit the compound to interact with the PLSCR polypeptide; and comparing the cellular response in a cell incubated with the compound with the response of a cell not incubated with the compound. Wherein a difference in response is indicative of a compound that modulates PLSCR activity. the modulation may be an inhibition of or stimulation of Phospholipid Scramblase expression.

The invention also includes a method of treating a disorder in a subject associated with Phospholipid Scramblase polypeptide activity. The method includes administering to a subject in need thereof a therapeutically effective amount of a compound that modulates a Phospholipid Scramblase polypeptide activity.

The invention also provides a method of diagnosis of a subject having or at risk of having a Phospholipid Scramblase-related disorder. The method includes detecting in the subject a level or activity of a Phospholipid Scramblase polypeptide that is different from the level or activity in a normal subject, thereby diagnosing a subject having or risk of having a Phospholipid Scramblase-related disorder.

The invention further provides a method of increasing or extending the viability of mammalian cells or tissues by inhibiting the expression of a Phospholipid Scramblase polynucleotide within the cell or tissue.

In yet another embodiment of the invention, there is provided a method of treating a subject having or at risk of having a disorder associated with a Phospholipid Scramblase polypeptide or polynucleotide. The method includes introducing into a subject a polynucleotide encoding the Phospholipid Scramblase polypeptide operatively linked to a regulatory sequence, thereby treating the subject.

### BRIEF DESCRIPTION OF THE DRAWINGS

FIG. 1 shows the nucleotide sequence and predicted amino acid sequence of human Phospholipid Scramblase 1 (huPLSCR1) (SEQ ID NO: 1 and 2, respectively).

FIG. 2 shows the nucleotide sequence and predicted amino acid sequence of human Phospholipid Scramblase 2 (huPLSCR2) (SEQ ID NO:3 and 4, respectively).

FIG. 3 shows the nucleotide sequence and predicted amino acid sequence of human Phospholipid Scramblase 3 (huPLSCR3) (SEQ ID NO:5 and 6, respectively).

FIGS. 4A and 4B show the nucleotide sequence and predicted amino acid sequence of human Phospholipid Scramblase 4 (huPLSCR4) (SEQ ID NO:7 and 8, respectively).

FIG. 5 shows the nucleotide sequence and predicted amino acid sequence of mouse Phospholipid Scramblase 1 (muPLSCR1) (SEQ ID NO:9 and 10, respectively).

FIG. 6 shows the nucleotide sequence and predicted amino acid sequence of mouse Phospholipid Scramblase 2 (muPLSCR2) (SEQ ID NO: 11 and 12, respectively).

FIG. 7 shows the nucleotide sequence and predicted amino acid sequence of mouse Phospholipid Scramblase 3 (muPLSCR3) (SEQ ID NO:13 and 14, respectively).

FIG. 8. shows the nucleotide sequence and predicted amino acid sequence of mouse Phospholipid Scramblase 4 (muPLSCR4) (SEQ ID NO: 15 and 16, respectively).

FIG. 9 shows the aligned protein sequence of the four known members of the human phospholipid scramblase gene family. Solid lines indicate PPxY motifs (not found in PLSCR2), dotted lines indicate residues identified as Ca²⁺ binding sites in PLSCR1; dashed lines indicate putative membrane-spanning segment.

FIG. 10 shows the predicted open reading frame of mouse PLSCR1 aligned against human PLSCR1. Alignment performed with GeneStream Align program (74.8% identity). Note conserved PPxY motif (solid box), Ca²⁺ binding segment (dotted box) and predicted transmembrane domain (dashed box).

FIG. 11 shows gene structure of the HuPLSCR1 gene. Top: Schematic showing location of intron-exon borders within the HuPLSCR1 genomic sequence of approx. 28 kb. Exons are represented by vertical lines. Below: Location of exons within the cDNA are indicated by arrows. Genomic sequence for HuPLSCR1 deposited under GenBank accession no. AF224492.

Four putative ISRE-like elements (filled boxes) located between 4120 bp and +60 bp of the 5′flanking region and first untranslated exon of PLSCR1 gene are depicted in linear map (top): #1=(−3815)gaaaagaGAATcc(−3800); #2=(−2733)acaaaaaGAAAgc−2721; #3=(−2519aaaaacaGAAAcc(−2497); #4=(+21)ggaaaagGAAAcc(+35). Arrow denotes transcription initiation site. Sequence spanning these various putative ISRE-like elements were selectively deleted by PCR and the truncated PLSCR1 DNA cloned into pGL3-luciferase reporter vector as described in Materials & Methods. Daudi cells were then co-transfected with β-galactosidase-pSV (as transfection efficiency control) and these PLSCR1-pGL3-luciferase plasmids containing the following insertions of PLSCR1 5′ genomic DNA: −4120 bp to +60 bp (spanning #1-4); −3307 bp to +60 bp (spanning #1-3); −2277 bp to +60 bp (spanning #1 only); −4120bp to +18 bp (spanning #2-4); and pGL3 vector without insert (vector). After 24 h transfection, either 0 (solid bars) or 1,000 IU/ml (open bars) IFN-α2a was added to the cell cultures, and 18 h later, the cells were harvested for measurement of luciferase and β-galactosidase activities (Materials & Methods). Bar graph reports ratio of luciferase/β-galactosidase activities measured at 18 h. Error bars denote mean ±SEM (n=3). Data of single experiment, representative of three experiments so performed. The average IFN-induced increase (mean±SD) obtained for each reporter construct from the combined data of all 3 experiments was 3.9±0.4 (insert spanning #1-4); 5.3±1.0 (insert spanning #1-3); 5.2±0.8 (insert spanning #1); 0.8±0.1 (insert spanning #2-4); 0.8±0.2 (vector control).

FIG. 12 shows the Analysis of genomic 5′flanking sequence of HuPLSCR1. Shown are consensus promoters and putative binding sites for transcriptional activators in 5′ flanking sequence and first untranslated exon of HuPLSCR1 gene (GenBank Accession AF153715) as predicted by MatInspector V2.2.

FIG. 13 shows the alignment of amino acid sequences of four HuPLSCR homologues. Alignment was performed using Clustal W (20). Identical amino acids are shown on black, and conservative substitutions on grey background. cDNA sequences deposited in GenBank under following accession numbers: HuPLSCR1 (human phospholipid scramblase), AF098642; HuPLSCR2, AF159441; HuPLSCR3, AF159442; HuPLSCR4, AF199023.

### DETAILED DESCRIPTION OF THE INVENTION

The present invention provides polynucleotides encoding a Phospholipid Scramblase polypeptide. These polynucleotides include DNA, cDNA and RNA sequences which encode Phospholipid Scramblase polypeptides, as well as splice variants of these sequences, allelic variants of these sequences, and homologous or orthologous variants of these sequences. It is understood that all polynucleotides encoding all or a portion of Phospholipid Scramblase polypeptides are also included herein, as long as they encode a polypeptide with Phospholipid Scramblase polypeptide activity. Such polynucleotides include naturally occurring, synthetic, and intentionally manipulated polynucleotides. For example, Phospholipid Scramblase polynucleotides may be subjected to site-directed mutagenesis. The polynucleotide sequence for Phospholipid Scramblase polypeptide also includes antisense sequences. The polynucleotides of the invention include sequences that are degenerate as a result of the genetic code. There are 20 natural amino acids, most of which are specified by more than one codon. Therefore, all degenerate nucleotide sequences are included in the invention as long as the amino acid sequence of Phospholipid Scramblase polypeptide encoded by the nucleotide sequence is functionally unchanged.

IFNs are a family of pleiotropic cytokines responsible for providing vertebrates with innate immunity against a wide-range of viruses and other microbial pathogens. In addition, IFNs have anti-tumor activities due to their anti-proliferative, apoptotic and immunoregulatory properties. Type I IFNs (14 subtypes of IFN-α, IFN-β and IFN-ω) are induced in most cell types in response to virus infections whereas type II IFN (IFN-γ) is induced in T lymphocytes and NK cells in response to immune and inflammatory stimulation. Type I IFNs are encoded in a gene cluster in human chromosome 9 while the IFN-γ gene maps to chromosome 12. IFNs bind to specific cell receptors activating JAK/STAT signaling pathways to the IFN-regulated genes. Although type I IFNs are not structurally related to type II IFN, both induce similar biological responses due to partially overlapping JAK/STAT signal transduction pathways. For instance, both type I and II IFNs induce an antiviral state, inhibit cell proliferation and cause major histocompatibility antigen (MHC) class I induction. On the other hand, IFN-γ possesses unique immune system functions by activating macrophages and inducing MHC class II through transcription factor CIITA.

The antiviral activities of IFNs are due in part to the induction of three biochemical pathways, the 2-5A system, protein kinase PKR and the Mx proteins. The 2-5A system is a regulated RNA decay pathway in which IFN-induced 2-5A synthetases polymerize ATP into 2′,5′-linked oligoadenylates (2-5A) in response to viral dsRNA. Our results have shown that 2-5A activates RNase L causes single-stranded RNA degradation, suppressing virus replication and promoting apoptosis. PKR is a dsRNA-dependent protein kinase that phosphorylates protein synthesis factor, eIF2-α, inhibiting protein synthesis, suppressing viral replication and causing apoptosis. PKR is also a regulator of transcription factors, including NF-κB. The Mx proteins are large GTPases in the dynamin superfamily that interfere with the movement of subviral particles in cells, suppressing replication of some negative RNA stranded viruses. Furthermore, it has been known for the last twenty years that the maturation and release of membrane bound viruses is inhibited by the IFNs. Accordingly, the release and/or maturation of vesicular stomatitis virus (VSV), retroviruses including MuLV and lentiviruses (HIV) are suppressed by IFN treatment. However, the molecular pathway responsible for this anti-viral function of IFNs is unknown. Preliminary data in this application suggest that these effects of IFN are mediated in part at the plasma membrane, potentially through IFN induction of PLSCR1.

The anti-proliferative activity of IFN, like the anti-viral pathways, involve multiple mechanisms. IFN treatment of cells leads to an accumulation of cells at the G1/S boundary of the cell cycle. Cell cycle factors targeted by IFNs include c-myc, pRB, cyclin D3 and cdc25A. For instance, IFN treatment of human lymphoblastoid Daudi cells causes inhibition of c-myc transcription, possibly by inhibiting the E2F transcription factor. IFN causes reductions in Rb phosphorylation by inhibition of cdk4 and cdk6, leading to growth inhibition of Daudi cells. The IFN inducible proteins, PKR and RNase L, are implicated in both the anti-proliferative and apoptotic activities of IFN. In addition, several genes have been cloned which, when downregulated, suppress the growth inhibitory or apoptotic activities of IFN-γ. The technical knock out (TKO) strategy led to the cloning of five novel genes for IFN-γ death-associated proteins, DAP-1 to -5, and the identification of two other genes encoding thioredoxin and cathepsin D protease. Furthermore, we have identified, using gene chips, several apoptotic genes to be IFN regulated, including BAK, fas, and HIFα.

All types of IFNs have the ability to enhance MHC class I expression leading to development of CD8⁺ T cell responses. On the other hand, IFN-γis responsible for inducing MHC class II leading to CD4⁺ T cell responses. The IFNs also enhance antigen processing and presentation by inducing genes for proteasome subunits (LMP2, LMP7, and MECL1) and TAP1 and TAP2 responsible for transporting peptides to the endoplasmic reticulum where they bind to MHC chains. IFN-γ promotes development of CD4+T cells into Th1 responsible for cell-mediated immunity and delay hypersensitivity responses. In addition, IFN-γ plays the predominant role in activating the cytocidal activities of macrophages. Finally, IFNs regulate humoral immunity by controlling immunoglobulin (Ig) secretion and Ig heavy-chain switching.

Despite over forty years of research on IFNs, there are substantial gaps in our understanding of the molecular mechanisms that are responsible for the biological effects of IFNs. For example, triply deficient mice lacking RNase L, PKR and Mxl can still mount a very substantial residual anti-viral effect in response to IFN-α. As will be detailed in SECTION C, we have recently identified phospholipid scramblase-1 (PLSCR1)—a plasma membrane protein—as a new member of the IFN-regulated gene family. The purpose of the proposed studies is to gain an understanding of the cellular and plasma membrane changes induced through IFN's transcriptional upregulation of PLSCR1, and how these changes mediated through PLSCR1 potentially relate to the biologic activity of IFN in vivo.

The plasma membrane PL of all mammalian cells are normally asymmetrically distributed: the aminoPL, including phosphatidylserine (PS) & phosphatidylethanolamine (PE) reside almost exclusively in the inner membrane leaflet, whereas the outer leaflet is enriched in neutral polar PL, including phosphatidylcholine (PC) and sphingomyelin (SM). Although this asymmetric distribution of plasma membrane PL was identified in the 1970's, it is only recently that experimental evidence establishing biological function has accumulated. It is now well-recognized that the transmembrane orientation of plasma membrane PL is central to the regulation of surface-localized enzyme reactions of both the complement and coagulation systems of blood plasma and to the recognition and phagocytic clearance of injured, aged or apoptotic cells (see below). It is also now generally accepted that the maintenance of PL asymmetry arises through the activity of a specific transmembrane PL “flippase” with specificity for the headgroups of aminoPL. This aminoPL-translocase has been shown to selectively and vectorially transport PS (>PE), but not neutral PL such as PC or SM, from outer to inner leaflets of the plasma membrane in a process that is dependent on millimolar concentrations of both Mg²⁺ and ATP. Data from a number of laboratories suggest that aminoPL-translocase is a specific P-type Mg²⁺-ATPase, possibly related to yeast DRS2 gene.

In circumstances of cell activation, cell injury, or in response to known apoptotic stimuli there is an extensive remodeling of the plasma membrane PL that results in rapid egress of PS and PE to the cell surface. Coincident with movement of aminoPL to the cell surface, there is accelerated inward flip of PC and SM from outer to inner leaflet, ultimately collapsing the normal compositional asymmetry across the plasma membrane.

The cellular mechanism(s) underlying this triggered transbilayer movement of plasma membrane PL remains unresolved. A number of studies have demonstrated that simple inactivation of the active inward transport of PS & PE through aminoPL-translocase (e.g. by metabolic depletion of cellular ATP, or by incubation with ATPase inhibitors), does not in itself collapse plasma membrane PL asymmetry nor result in significant cell-surface exposure of the aminoPL, as long as normally low [Ca²⁺] in the cytosol is maintained. This effect of intracellular Ca²⁺ on transbilayer migration of membrane PL is abrogated by trypsin, and activated by acidification (pH<6.5) in absence of Ca²⁺, implying that transbilayer migration of PL is initiated through interaction of Ca²⁺ with His, or carboxylate (Asp or Glu) residues of an endofacial membrane protein.

During the past three years, we have made considerable progress in defining potential molecular mechanisms underlying this Ca²⁺-induced transbilayer movement of plasma membrane PL (research conducted under R01 HL36946). We reported the purification and characterization of an integral erythrocyte membrane protein [designated “PL scramblase; hu PLSCR1”] that, when reconstituted in liposomes, mediates a Ca²⁺-dependent and pH-dependent accelerated transbilayer movement of all PL, mimicking the reorganization of plasma membrane PL observed either upon elevation of [Ca²⁺]_{c} or upon acidification of the cytosol. There is also evidence that the same protein mediates similar function in platelets. The properties of this protein in PL bilayer membranes indicate that PLSCR1 is responsible for accelerated transbilayer movement of PS and other plasma membrane PL in all cells and tissues exposed to elevated [Ca²⁺]_{c}, arising as a consequence of immune injury or agonist-induced cell activation, and potentially, during cell senescence (see below)

The deduced sequence of human PLSCR1 reveals a proline-rich acidic protein (35.1 kD; pKa=4.85) with a single predicted transmembrane domain near the C-terminus. There is also a single potential protein kinase C phosphorylation site (Thr¹⁶¹) and an apparent EF-hand related Ca²⁺-binding motif (see below). Analysis of the cDNA-derived protein sequence predicts a strongly preferred inside-to-outside orientation of the predicted 19-residue transmembrane domain, consistent with a type 2 plasma membrane protein. Thus, the bulk of the protein is predicted to extend from the cytoplasmic membrane leaflet, leaving a short extracellular tail. The predicted orientation of this protein is consistent with the anticipated topology of PL scramblase in erythrocytes and platelets, where the lipid-mobilizing function is responsive to Ca²⁺ or to acidification (pH<6.5) only at the endofacial surface of the plasma membrane. Northern blotting revealed that PLSCR1 mRNA was present in a variety of hematological and non-hematological cells and tissues. We have identified the residues in PLSCR1 that function in binding Ca²⁺ and our data suggest that the activity of PLSCR1 is also regulated post-translationally through palmitoylation at one or more Cys thiols. We have recently identified three additional members of the PL scramblase gene family (hu PLSCR 2-4) and the putative PL scramblase orthologues in mouse (mu PLSCR1-4) have been identified.

We have shown that PLSCR1 is induced in IFN treated cells where it localizes to the plasma membrane, the site of budding for these viruses. The N-terminal regions of hu and mu PLSCR1 share with diverse types of membrane bound viruses late function PPxY motifs required for release of virus particles from cells. PPxY motifs in PLSCR1 suppress virus budding by competing with the viral M or Gag proteins for binding to cellular WW domain proteins. This is the first example of host mimicry of a viral protein for the purpose of suppressing virus infections.

The consensus sequence, PPxY, is implicated in the virus budding process for rhabdoviruses, filoviruses and retroviruses where it is present in the viral matrix (M) or Gag proteins. For example, mutations in the PPxY motif of the VSV matrix (M) protein prevent budding. The functional homologue of the PPxY motif in the HIV and visna virus Gag proteins is P(S/T)APP. These different late function motifs can often substitute for each other in promoting budding of chimeric particles. Remarkably, many of the same viruses that are inhibited by IFN at the level of virus maturation and release also contain the PPxY or related motifs in M or Gag proteins, notably rhabdoviruses, retroviruses and lentiviruses. In retroviruses, the motif is termed the late (L) domain which in the Rous sarcoma virus Gag protein consists of a PPPPY sequence near the N-terminus. In filoviruses, the VP40 (presumptive matrix) proteins of Ebola (Zaire strain) and Marburg virus (Popp strain) contain the sequence PPEY and PPPY, respectively. In VSV and Rabies viruses, the motif in the M protein is PPPY and PPEY. The L domain and PPxY motifs function by binding to cellular WW domain proteins required for virus budding. A defining feature of WW domains is the presence of two conserved tryptophan (W) residues in a 38 amino acid repeating unit. For instance, the WW domain in the Yes-associated protein (YAP) was shown to interact with the p2b region of the RSV Gag protein and with the PPxY motifs in the VSV and rabies virus M proteins in vitro.

Cellular as well as viral functions of PLSCR1 could be mediated by interactions between the PPxY motif in PLSCR1 and cellular WW domain proteins. For instance, putative WW domain proteins could modify PLSCR1 or otherwise affect PL scrambling. In addition, the N-terminal segment of the PLSCR1 polypeptide also contains PXXP motifs which may serve as potential binding sites for proteins containing SH3 domains.

A potential link between PLSCR1 gene expression and neoplastic cell transformation was recently suggested by Kasukabe and associates. They describe a gene transcript (designated NOR1) that is markedly down-regulated in transformed murine monocytic cell lines relative to its expression in normal blood monocytes, and, a 5′-truncated form of this same transcript (designated TRA1) expressed only in leukemogenic mouse monocytic cell lines (but not expressed in normal monocyte or non-leukemogenic monocytic cell lines). Butyrate induction of monocytes to macrophages was accompanied by induced expression of the NOR1 transcript²⁸. They suggest that the truncated TRA1 gene product is associated with leukemogenesis in vivo, whereas increased NOR1 expression is associated with macrophage differentiation. Analysis of the open reading frame predicted by the NOR1 and TRA1 cDNA sequences reveals near-identity of protein sequence with hu PLSCR1, in overlapping portions of each polypeptide. This suggests that NOR1 (expressed in normal mouse monocytes and other tissues) is the murine orthologue of human PLSCR1 (mu PLSCR1) whereas the TRA1 gene product, found only in leukemogenic cell lines, is a truncated form of mu PLSCR1 that arises through alternative splicing (deleting exons 1-5). Down-regulation of wild-type mu PLSCR1 (i.e. NOR1) in transformed monocytes and the de novo expression of the alternatively-spliced, truncated form of this protein (i.e., TRA1) in only leukemogenic subclones, suggests that NOR1 (and thus presumably PLSCR1) is required for normal cell senescence, whereas mutant TRA1 might promote leukemogenic potential, potentially as a dominant-negative PL scramblase inhibitor.

As was noted, we now have shown that the expression of hu PLSCR1 is markedly upregulated by IFN. PLSCR1 directly contributes to the antiproliferative action of IFN and provides a causal explanation for the observed association of aberrant NOR1/TRA1 gene expression in transformed and leukemogenic cell lines.

The invention includes a functional Phospholipid Scramblase polypeptide, and functional fragments thereof. As used herein, the term “functional polypeptide” refers to a polypeptide which possesses a biological function or activity which is identified through a defined functional assay and which is associated with a particular biologic, morphologic, or phenotypic alteration in the cell. Functional fragments of the Phospholipid Scramblase polypeptide, include fragments of Phospholipid Scramblase which retain the activity of Phospholipid Scramblase. Smaller peptides containing the biological activity of Phospholipid Scramblase are included in the invention. The biological function, for example, can vary from a polypeptide or polynucleotide fragment as small as an epitope to which an antibody molecule can bind to a large polypeptide which is capable of participating in the characteristic induction or programming of phenotypic changes within a cell. A “functional polynucleotide” denotes a polynucleotide which encodes a functional polypeptide as described herein.

“Isolated” means altered “by the hand of man” from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide separated from the coexisting materials of its natural state is “isolated”, as the term is employed herein.

“Polynucleotide” generally refers to any polyribonucleotide or polydeoxribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

The present invention also specifically provides for mutant or disease-causing variants of the Phospholipid Scramblases. Because the nucleic acids of the invention may be used in a variety of diagnostic, therapeutic and recombinant applications, various subsets of the Phospholipid Scramblase sequences and combinations of the Phospholipid Scramblase sequences with heterologous sequences are also provided. For example, for use in allele specific hybridization screening or PCR amplification techniques, subsets of the Phospholipid Scramblase sequences, including both sense and antisense sequences, and both normal and mutant sequences, as well as intronic, exonic and untranslated sequences, are provided. Such sequences may comprise a small number of consecutive nucleotides from the sequences which are disclosed or otherwise enabled herein but preferably include at least 8-10, and more preferably 9-25, consecutive nucleotides from a Phospholipid Scramblase sequence. Other preferred subsets of the Phospholipid Scramblase sequences include those encoding one or more of the functional domains or antigenic determinants of the Phospholipid Scramblase polypeptides and, in particular, may include either normal (wild-type) or mutant sequences. The invention also provides for various nucleic acid constructs in which Phospholipid Scramblase sequences, either complete or subsets, are operably joined to exogenous sequences to form cloning vectors, expression vectors, fusion vectors, transgenic constructs, and the like.

Exemplary polynucleotides encoding a Phospholipid Scramblase polypeptide are set forth as SEQ ID NOs:1, 3, 5, 7, 9, 11, 13 and 15 or fragments thereof The term “polynucleotide”, “nucleic acid”, “nucleic acid sequence”, or “nucleic acid molecule” refers to a polymeric form of nucleotides at least 10 bases in length. By “isolated polynucleotide” is meant a polynucleotide that is not immediately contiguous with both of the coding sequences with which it is immediately contiguous (one on the 5′ end and one on the 3′ end) in the naturally occurring genome of the organism from which it is derived. The term therefore includes, for example, a recombinant nucleic acid construct which is incorporated into a vector; into an autonomously replicating plasmid or virus; or into the genomic DNA of a prokaryote or eukaryote, or which exists as a separate molecule (e.g., a cDNA) independent of other sequences. The nucleotides of the invention can be ribonucleotides, deoxyribonucleotides, or modified forms of either nucleotide.

The phrases “nucleic acid” or “nucleic acid sequence,” as used herein, refer to an oligonucleotide, nucleotide, polynucleotide, or any fragment thereof, to DNA or RNA of genomic or synthetic origin which may be single-stranded or double-stranded and may represent the sense or the antisense strand, to peptide nucleic acid (PNA), or to any DNA-like or RNA-like material. In this context, “fragments” refers to those nucleic acid sequences which are greater than about 15 to 60 nucleotides in length, and more preferably are at least about 100 nucleotides, at least about 1000 nucleotides, or at least about 10,000 nucleotides in length. Examples of nucleic acid fragments include SEQ ID NO: 15 and nucleic acid sequences encoding SEQ ID NO: 16, for example. Such fragments may be included in fusion proteins, as inhibitors or mimetics of Phospholipid Scramblase polypeptides or immunogens.

The term “homology,” as used herein, refers to a degree of complementarity. There may be partial homology or complete homology. A partially complementary sequence that at least partially inhibits an identical sequence from hybridizing to a target nucleic acid is referred to as “substantially homologous.” The inhibition of hybridization of the completely complementary sequence to the target sequence may be examined using a hybridization assay (Southern or northern blot, solution hybridization, and the like) under conditions of reduced stringency. A substantially homologous sequence or hybridization probe will compete for and inhibit the binding of a completely homologous sequence to the target sequence under conditions of reduced stringency. This is not to say that conditions of reduced stringency are such that non-specific binding is permitted, as reduced stringency conditions require that the binding of two sequences to one another be a specific (i.e., a selective) interaction. The absence of non-specific binding may be tested by the use of a second target sequence which lacks even a partial degree of complementarity (e.g., less than about 30% homology or identity). In the absence of non-specific binding, the substantially homologous sequence or probe will not hybridize to the second non-complementary target sequence.

The terms “complementary” or “complementarity,” as used herein, refer to the natural binding of polynucleotides under permissive salt and temperature conditions by base pairing. For example, the sequence “A-G-T” binds to the complementary sequence “T-C-A.” Complementarity between two single-stranded molecules may be “partial,” such that only some of the nucleic acids bind, or it may be “complete,” such that total complementarity exists between the single stranded molecules. The degree of complementarity between nucleic acid strands has significant effects on the efficiency and strength of the hybridization between the nucleic acid strands. This is of particular importance in amplification reactions, which depend upon binding between nucleic acids strands, and in the design and use of peptide nucleic acid (PNA) molecules.

The phrases “percent identity” or “% identity” refer to the percentage of sequence similarity found in a comparison of two or more amino acid or nucleic acid sequences. Percent identity can be determined electronically, e.g., by using the MEGALIGN program (DNASTAR, Inc., Madison, Wis.). The MEGALIGN program can create alignments between two or more sequences according to different methods, e.g., the clustal method. (See, e.g., Higgins, D. G. and P. M. Sharp (1988) Gene 73:237-244.) The clustal algorithm groups sequences into clusters by examining the distances between all pairs. The clusters are aligned pairwise and then in groups. The percentage similarity between two amino acid sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no homology between the two amino acid sequences are not included in determining percentage similarity. Percent identity between nucleic acid sequences can also be counted or calculated by other methods known in the art, e.g., the Jotun Hein method. (See, e.g., Hein, J. (1990) Methods Enzymol. 183:626-645.) Identity between sequences can also be determined by other methods known in the art, e.g., by varying hybridization conditions.

In the present invention, the Phospholipid Scramblase polynucleotide sequences may be inserted into a recombinant expression vector. The term “expression vector” refers to a plasmid, virus or other vehicle known in the art that has been manipulated by insertion or incorporation of the Phospholipid Scramblase polynucleotide sequences. Such expression vectors contain a promoter sequence which facilitates the efficient transcription of the inserted genetic sequence of the host. The expression vector typically contains an origin of replication, a promoter, as well as specific genes which allow phenotypic selection of the transformed cells. Vectors suitable for use in the present invention include, but are not limited to the T7-based expression vector for expression in bacteria (Rosenberg, et al. Gene, 56:125 (1987), the pMSXND expression vector for expression in mammalian cells (Lee and Nathans (1988) J. Bio. Chem., 263:3521 ) and baculovirus-derived vectors for expression in insect cells. The DNA segment can be present in the vector operably linked to regulatory elements, for example, a promoter (e.g., T7, metallothionein I, or polyhedrin promoters). Such expression vectors can be utilized, for example, to produce a protein of the invention in vitro. The expression vector is introduced into a suitable host cell and cultured under conditions that allow expression of the polynucleotide. Expression vectors are also useful, for example, for in vivo uses such as gene therapy.

In general, an expression vector contains the expression elements necessary to achieve, for example, sustained transcription of the nucleic acid molecule, although such elements also can be inherent to the nucleic acid molecule cloned into the vector. In particular, an expression vector contains or encodes a promoter sequence, which can provide constitutive or, if desired, inducible expression of a cloned nucleic acid sequence, a poly-A recognition sequence, and a ribosome recognition site, and can contain other regulatory elements such as an enhancer, which can be tissue specific. The vector also contains elements required for replication in a prokaryotic or eukaryotic host system or both, as desired. Such vectors, which include plasmid vectors and viral vectors such as bacteriophage, baculovirus, retrovirus, lentivirus, adenovirus, vaccinia virus, semliki forest virus and adeno-associated virus vectors, are well known and can be purchased from a commercial source (Promega, Madison Wis.; Stratagene, La Jolla Calif.; GIBCO/BRL, Gaithersburg Md.) or can be constructed by one skilled in the art (see, for example, Meth. Enzymol., Vol. 185, D. V. Goeddel, ed. (Academic Press, Inc., 1990); Jolly, Canc. Gene Ther. 1:51-64 (1994); Flotte, J. Bioenerg. Biomemb. 25:37-42 (1993); Kirshenbaum et al., J. Clin. Invest 92:381-387 (1993), which is incorporated herein by reference).

In particular, an expression vector contains a promoter sequence, which can provide constitutive or, if desired, inducible expression of the encoding nucleic acid molecule, and a poly-A recognition sequence, and can contain other regulatory elements such as an enhancer, which can be tissue specific.

Similarly, a eukaryotic expression vector can include, for example, a heterologous or homologous RNA transcription promoter for RNA polymerase binding, a polyadenylation signal located downstream of the coding sequence, an AUG start codon in the appropriate frame and a termination codon to direct detachment of a ribosome following translation of the transcribed mRNA

In general, expression vectors containing promoter sequences which facilitate the efficient transcription of the inserted genetic sequence are used in connection with the host. As described above, biologically functional viral or plasmid DNA vectors capable of expression and replication in a host are known in the art. Such vectors are used to incorporate encoding DNA sequences of the invention. Expression vectors typically contain an origin of replication, a promoter, and a terminator, as well as specific genes that are capable of providing phenotypic selection of the transformed cells.

Polynucleotide sequences encoding Phospholipid Scramblase polypeptides can be expressed in either prokaryotes or eukaryotes. Hosts can include microbial, yeast, insect and mammalian organisms. Methods of expressing DNA sequences having eukaryotic or viral sequences in prokaryotes are well known in the art. Biologically functional viral and plasmid DNA vectors capable of expression and replication in a host are known in the art. Such vectors are used to incorporate DNA sequences of the invention.

Methods that are well known to those skilled in the art can be used to construct expression vectors containing the Phospholipid Scramblase polypeptide coding sequence and appropriate transcriptional/translational control signals. These methods include in vitro recombinant DNA techniques, synthetic techniques, and in vivo recombinant/genetic techniques. See, for example, the techniques described in Maniatis, et al., 1989 Molecular cloning, A Laboratory Manual, Cold Spring Harbor Laboratory, N.Y.

A Phospholipid Scramblase polypeptide or a fragment thereof, can be encoded by a recombinant or non-recombinant nucleic acid molecule and expressed in a cell. Preparation of a Phospholipid Scramblase polypeptide by recombinant methods provides several advantages. In particular, the nucleic acid sequence encoding the Phospholipid Scramblase polypeptide can include additional nucleotide sequences encoding, for example, peptides useful for recovering the Phospholipid Scramblase polypeptide from the host cell. A Phospholipid Scramblase polypeptide can be recovered using well known methods, including, for example, precipitation, gel filtration, ion exchange, reverse-phase, or affinity chromatography (see, for example, Deutscher et al., “Guide to Protein Purification” in Meth. Enzymol., Vol. 182, (Academic Press, 1990)). Such methods also can be used to purify a fragment of a Phospholipid Scramblase polypeptide, for example, a particular binding sequence, from a cell in which it is naturally expressed.

A recombinant nucleic acid molecule encoding a Phospholipid Scramblase polypeptide or a fragment thereof can include, for example, a protease site, which can facilitate cleavage of the Phospholipid Scramblase polypeptide from a non-Phospholipid Scramblase polypeptide sequence, for example, a tag peptide, secretory peptide, or the like. As such, the recombinant nucleic acid molecule also can encode a tag peptide such as a polyhistidine sequence, a FLAG peptide (Hopp et al., Biotechnology 6:1204 (1988)), a glutathione S-transferase polypeptide or the like, which can be bound by divalent metal ions, a specific antibody (U.S. Pat. No. 5,011,912), or glutathione, respectively, thus facilitating recovery and purification of the Phospholipid Scramblase polypeptide comprising the peptide tag. Such tag peptides also can facilitate identification of the Phospholipid Scramblase polypeptide through stages of synthesis, chemical or enzymatic modification, linkage, or the like. Methods for purifying polypeptides comprising such tags are well known in the art and the reagents for performing such methods are commercially available.

A nucleic acid molecule encoding a Phospholipid Scramblase polypeptide can be engineered to contain one or more restriction endonuclease recognition and cleavage sites, which can facilitate, for example, substitution of an element of the Phospholipid Scramblase polypeptide such as the selective recognition domain or, where present, a spacer element. As such, related Phospholipid Scramblase polypeptides can be prepared, each having a similar activity, but having specificity for different function-forming contexts. A restriction endonuclease site also can be engineered into (or out of) the sequence coding a peptide portion of the Phospholipid Scramblase polypeptide, and can, but need not change one or more amino acids encoded by the particular sequence. Such a site can provide a simple means to identify the nucleic acid sequence, based on cleavage (or lack of cleavage) following contact with the relevant restriction endonuclease, and, where introduction of the site changes an amino acid, can further provide advantages based on the substitution.

In another embodiment, the present invention provides a substantially pure Phospholipid Scramblase polypeptide. The present invention provides for substantially pure protein preparations including polypeptides comprising or derived from the Phospholipid Scramblase polypeptides. The Phospholipid Scramblase polypeptide sequences of the invention include the specifically disclosed sequences, variants of these sequences resulting from alternative MRNA splicing, allelic variants of these sequences, mutations of these sequences and homologous or orthologous variants of these sequences.

As used herein, the term “substantially pure” refers to Phospholipid Scramblase polypeptide that is substantially free of other proteins, lipids, carbohydrates or other materials with which it is naturally associated. One skilled in the art can purify Phospholipid Scramblase polypeptides using standard techniques for protein purification. For example, the substantially pure polypeptide will yield a single major band on a non-reducing polyacrylamide gel. The purity of a Phospholipid Scramblase polypeptide can also be determined by amino-terminal amino acid sequence analysis or other methods known in the art.

A functional Phospholipid Scramblase polypeptide includes a polypeptide as set forth in SEQ ID NOs:4, 6, 8, 14 and 16 and variations thereof, including conservative variations, as an illustrative polypeptide, as well as mutant or disease-causing variants of the Phospholipid Scramblases. The terms “conservative variation” and “substantially similar” as used herein denotes the replacement of an amino acid residue by another, biologically similar residue. Examples of conservative variations include the substitution of one hydrophobic residue such as isoleucine, valine, leucine or methionine for another, or the substitution of one polar residue for another, such as the substitution of arginine for lysine, glutamic acid for aspartic acid, or glutamine for asparagine, and the like. The terms “conservative variation” and “substantially similar” also include the use of a substituted amino acid in place of an unsubstituted parent amino acid provided that antibodies raised to the substituted polypeptide also immunoreact with the unsubstituted polypeptide. Modifications include stabilization of Phospholipid Scramblase or of the biological activity thereof.

As used herein, the terms “protein” or “polypeptide” are used in the broadest sense to mean a sequence of amino acids that can be encoded by a cellular gene or by a recombinant nucleic acid sequence or can be chemically synthesized. Because the proteins of the invention may be used in a variety of diagnostic, therapeutic and recombinant applications, various subsets of the Phospholipid Scramblase polypeptide sequences and combinations of the Phospholipid Scramblase polypeptide sequences with heterologous sequences are also provided. In some cases, the term “peptide” is used in referring to a portion of an amino acid sequence encoding a full length protein. A polypeptide can be a complete, full length gene product, which can be a core protein having no amino acid modifications or can be a post-translationally modified form of a protein such as a phosphoprotein, glycoprotein, proteoglycan, lipoprotein and nucleoprotein. The terms “amino acid” or “amino acid sequence,” as used herein, refer to an oligopeptide, peptide, polypeptide, or protein sequence, or a fragment of any of these, and to naturally occurring or synthetic molecules. In this context, “fragments”, “immunogenic fragments”, or “antigenic fragments” refer to fragments of Phospholipid Scramblase polypeptide which are preferably about 5 to about 15 amino acids or about 5 to 50 amino acids in length, but which can be longer, and which retain some biological activity or immunological activity of Phospholipid Scramblase.

Where “amino acid sequence” is recited herein to refer to an amino acid sequence of a naturally occurring protein molecule, “amino acid sequence” and like terms are not meant to limit the amino acid sequence to the complete native amino acid sequence associated with the recited protein molecule. For example, for use as immunogens or in binding assays, subsets of the Phospholipid Scramblase polypeptide sequences, including both normal and mutant sequences, are provided. Such protein sequences may comprise a small number of consecutive amino acid residues from the sequences which are disclosed or otherwise enabled herein but preferably include at least 4-8, and preferably at least 9-15 consecutive amino acid residues from a Phospholipid Scramblase polypeptide sequence. Other preferred subsets of the Phospholipid Scramblase polypeptide sequences include those corresponding to one or more of the functional domains or antigenic determinants of the Phospholipid Scramblase polypeptode and, in particular, may include either normal (wild-type) or mutant sequences. The invention also provides for various protein constructs in which Phospholipid Scramblase sequences, either complete or subsets, are joined to exogenous sequences to form fusion proteins and the like. In accordance with these embodiments, the present invention also provides for methods of producing all of the above described proteins which comprise, or are derived from, the Phospholipid Scramblases.

As used herein, the term “biologically active,” refers to a protein having structural, regulatory, or biochemical functions of a naturally occurring molecule. Likewise, “immunologically active” refers to the capability of the natural, recombinant, or synthetic Phospholipid Scramblase, or of any oligopeptide thereof, to induce a specific immune response in appropriate animals or cells and to bind with specific antibodies.

The invention also provides antibodies that bind to Phospholipid Scramblase polypeptides or fragments thereof of the invention. Such antibodies may prevent interactions of the Phospholipid Scramblase polypeptides with other proteins. The term “antibody” as used in this invention includes intact molecules as well as fragments thereof, such as Fab, F(ab′)2, and Fv which are capable of binding to an epitopic determinant present in an invention polypeptide. Such antibody fragments retain some ability to selectively bind with its antigen or receptor. Binding of antibodies to Phospholipid Scramblase polypeptides may interfere with trans-bilayer movement of membrane phospholipids. Antibody which consists essentially of pooled monoclonal antibodies with different epitopic specificities, as well as distinct monoclonal antibody preparations, and polyclonal preparations are provided.

As is mentioned above, antigens that can be used in producing Phospholipid Scramblase polypeptide-specific antibodies include Phospholipid Scramblase polypeptides or Phospholipid Scramblase polypeptide fragments. The polypeptide or peptide used to immunize an animal can be obtained by standard recombinant, chemical synthetic, or purification methods. As is well known in the art, in order to increase immunogenicity, an antigen can be conjugated to a carrier protein. Commonly used carriers include keyhole limpet hemocyanin (KLH), thyroglobulin, bovine serum albumin (BSA), and tetanus toxoid. The coupled peptide is then used to immunize the animal (e.g., a mouse, a rat, or a rabbit). In addition to such carriers, well known adjuvants can be administered with the antigen to facilitate induction of a strong immune response.

The antibodies of the invention can be used in any subject in which it is desirable to administer in vitro or in vivo immunodiagnosis or immunotherapy. The antibodies of the invention are suited for use, for example, in immunoassays in which they can be utilized in liquid phase or bound to a solid phase carrier. In addition, the antibodies in these immunoassays can be detectably labeled in various ways. Examples of types of immunoassays which can utilize antibodies of the invention are competitive and non-competitive immunoassays in either a direct or indirect format. Examples of such immunoassays are the radioimmunoassay (RIA) and the sandwich (immunometric) assay. Detection of the antigens using the antibodies of the invention can be done utilizing immunoassays which are run in either the forward, reverse, or simultaneous modes, including immunohistochemical assays on physiological samples. Those of skill in the art will know, or can readily discern, other immunoassay formats without undue experimentation.

A method is provided for producing a polypeptide containing the amino acid sequence of SEQ ID NO:4, 6, 8, 10, 14, or 16 or fragments thereof, including culturing the host cell under conditions suitable for the expression of the polypeptide and recovering the polypeptide from the host cell culture.

In a further embodiment of the invention, there is a provided an isolated nucleic acid sequence comprising a non-coding regulatory sequence isolated upstream from a Phospholipid Scramblase gene, wherein the nucleic acid sequence contains at least one restriction site for cloning a heterologous nucleic acid sequence of interest. In one aspect of the invention, the nucleic acid sequence is operably linked to a heterologous nucleic acid sequence thereby forming a DNA construct.

The term “operably associated” refers to functional linkage between a promoter sequence and the structural gene regulated by the promoter nucleic acid sequence. The operably linked promoter controls the expression of the polypeptide encoded by the structural gene. The particular promoter selected should be capable of causing sufficient expression to result in the production of an effective amount of the structural gene product, e.g., Phospholipid Scramblase polypeptide. The promoters used in the vector constructs of the present invention may be modified, if desired, to affect their control characteristics.

Optionally, a selectable marker may be associated with the heterologous nucleic acid sequence, i.e., the structural gene operably linked to a promoter. As used herein, the term “marker” refers to a gene encoding a trait or a phenotype which permits the selection of, or the screening for, a cell or organism containing the marker. Preferably, the marker gene is a selector marker gene whereby transformed cells can be selected from among cells that are not transformed. A reporter gene whereby transformed cells can be identified from among cells that are not transformed can be used. Examples of suitable reporter genes include the glucuronisdase (GUS) gene and the luciferase (LUC) reporter gene. Other suitable marker genes and reporter genes will be known to those of skill in the art.

In one aspect of the invention, a method is provided for identifying compounds that modulates expression of a Phospholipid Scramblase polypeptide including incubating the compound with a cell expressing a Phospholipid Scramblase DNA construct under conditions sufficient to permit the compound to interact with the construct and detecting expression of the heterologous gene in the presence of the compound compared to expression in the absence of the compound.

The cell may be any cell of interest, including but not limited to neuronal cells, glial cells, cardiac cells, bronchial cells, uterine cells, testicular cells, liver cells, renal cells, intestinal cells, cells from the thymus and spleen, placental cells, endothelial cells, endocrine cells including thyroid, parathyroid, pituitary and the like, smooth muscle cells and skeletal muscle cells. The cell is exposed to conditions sufficient to activate calcium mobilization. The effect of the compound on the cellular response is determined, either directly or indirectly, and a cellular response is then compared with a cellular response of a control cell. A suitable control includes, but is not limited to, a cellular response of a cell not contacted with the compound. The term “incubating” includes conditions which allow contact between the test compound and the cell of interest.

When Phospholipid Scramblase polypeptide expression is of interest, the modulation can be an inhibition in Phospholipid Scramblase polypeptide expression or a stimulation in Phospholipid Scramblase polypeptide expression.

Compounds that modulate a cellular response can include peptides, peptidomimetics, polypeptides, pharmaceuticals, chemical compounds and biological agents, for example. Antibodies, trophic agents, and combinatorial compound libraries can also be tested using the method of the invention. One class of organic molecules, preferably small organic compounds having a molecular weight of more than 50 and less than about 2,500 Daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups.

The test agent may also be a combinatorial library for screening a plurality of compounds. Compounds such as peptides identified in the method of the invention can be further cloned, sequenced, and the like, either in solution of after binding to a solid support, by any method usually applied to the isolation of a specific DNA sequence Molecular techniques for DNA analysis (Landegren et al., Science 242:229-237, 1988) and cloning have been reviewed (Sambrook et al., Molecular Cloning: a Laboratory Manual, 2nd Ed.; Cold Spring Harbor Laboratory Press, Plainview, N.Y., 1998, herein incorporated by reference).

Candidate compounds are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc., to produce structural analogs. Candidate agents are also found among biomolecules including, but not limited to: peptides, saccharides, fatty acids, steroids, purines, pyrimidines, derivatives, structural analogs or combinations thereof.

A variety of other agents may be included in the screening assay. These include agents like salts, neutral proteins, e.g., albumin, detergents, etc. that are used to facilitate optimal protein-protein binding and/or reduce nonspecific or background interactions. Reagents that improve the efficiency of the assay, such as protease inhibitors, nuclease inhibitors, antimicrobial agents, and the like may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 10 h will be sufficient. Alternatively, appropriate screening assays may be cell based.

The term “modulate,” as it appears herein, refers to a change in the activity or level of Phospholipid Scramblase. For example, modulation may cause an increase or a decrease in polypeptide activity, binding characteristics, expression or any other biological, functional, or immunological properties of Phospholipid Scramblase. The term “modulate” envisions the increased expression of Phospholipid Scramblase polynucleotide when Phospholipid Scramblase is under-expressed. Alternatively, when a disorder is associated with under-expression of Phospholipid Scramblase polypeptide, a sense polynucleotide sequence (the DNA coding strand) encoding Phospholipid Scramblase polypeptide, or 5′ regulatory nucleotide sequences (i.e., promoter) of Phospholipid Scramblase in operable linkage with Phospholipid Scramblase polynucleotide can be introduced into a cell. Therefore, the present invention also provides gene therapy for the treatment of cell proliferative disorders which are mediated by Phospholipid Scramblase. Such therapy would achieve its therapeutic effect by introduction of the appropriate Phospholipid Scramblase polynucleotide which contains a Phospholipid Scramblase structural gene (sense), into cells of subjects having the disorder. Delivery of sense Phospholipid Scramblase polynucleotide constructs can be achieved using a recombinant expression vector such as a chimeric virus or a colloidal dispersion systems.

Detection of altered (decreased or increased) levels of a Phospholipid Scramblase polypeptide or altered activity can be accomplished by hybridization of nucleic acids isolated from a cell of interest with a Phospholipid Scramblase polynucleotide of the invention. Analysis, such as Northern Blot analysis, are utilized to measure expression of Phospholipid Scramblase polypeptide, such as to measure Phospholipid Scramblase polypeptide transcripts. Other standard nucleic acid detection techniques will be known to those of skill in the art. Detection of altered levels of Phospholipid Scramblase protein activity can also accomplished using assays designed to detect Phospholipid Scramblase polypeptide. For example, antibodies or peptides that specifically bind a Phospholipid Scramblase polypeptide can be utilized. Analyses, such as Western blot analysis, radioimmunoassay or immunohistochemistry, are then used to measure Phospholipid Scramblase polypeptide concentration qualitatively or quantitatively.

In another embodiment, the present invention provides transgenic non-human animal models for disorders associated with disruptions in an Phospholipid Scramblase gene. The transgenic animal is mammalian specifically mice. The animal models are produced by standard transgenic methods including microinjection, transfection, or by other forms of transformation of embryonic stem cells, zygotes, gametes, and germ line cells with vectors including genomic or cDNA fragments, minigenes, homologous recombination vectors, viral insertion vectors and the like. Suitable vectors include vaccinia virus, adenovirus, adeno-associated virus, retrovirus, liposome transport, neuraltropic viruses, Herpes simplex virus, and the like. The animal models may include transgenic sequences comprising or derived from Phospholipid Scramblases, including normal and mutant sequences, intronic, exonic and untranslated sequences, and sequences encoding subsets of Phospholipid Scramblases such as functional domains. The major types of animal models provided include animals in which, (a) a mutant version of one of that animal's Phospholipid Scramblase genes has been recombinantly introduced into the genome of the animal as an additional gene, under the regulation of either an exogenous or an endogenous promoter element, and as either a minigene or a large genomic fragment; and/or in which a mutant version of one of that animal's Phospholipid Scramblase genes has been recombinantly substituted for one or both copies of the animal's homologous Phospholipid Scramblase gene by homologous recombination or gene targeting; (b) “Knock-out” animals in which one or both copies of one of the animal's Phospholipid Scramblase genes have been partially or completely deleted by homologous recombination or gene targeting, or have been inactivated by the insertion or substitution by homologous recombination or gene targeting of exogenous sequences. In a preferred embodiment, a transgenic mouse is homozygous or heterozygous for a disruption of an endogenous Phospholipid Scramblase polypeptide gene, for example, a gene that contains the polynucleotide sequence set forth in SEQ ID NO:9, 11, 13 or 15.

In a preferred embodiment of the invention, there is provided a transgenic mouse having a transgene that expresses a Phospholipid Scramblase protein polynucleotide chromosomally integrated into the germ cells of the animal. Animals are referred to as “transgenic” when such animal has had a heterologous DNA sequence, or one or more additional DNA sequences normally endogenous to the animal (collectively referred to herein as “transgenes”) chromosomally integrated into the germ cells of the animal. The transgenic animal (including its progeny) will also have the transgene fortuitously integrated into the chromosomes of somatic cells.

Various methods to make the transgenic mice of the subject invention can be employed. Generally speaking, three such methods may be employed. In one such method, an embryo at the pronuclear stage (a “one cell embryo”) is harvested from a female and the transgene is microinjected into the embryo, in which case the transgene will be chromosomally integrated into both the germ cells and somatic cells of the resulting mature animal. In another such method, embryonal stem cells are isolated and the transgene incorporated therein by electroporation, plasmid transfection or microinjection, followed by reintroduction of the stem cells into the embryo where they colonize and contribute to the germ line. Methods for microinjection of mammalian species is described in U.S. Pat. No. 4,873,191. In yet another such method, embryonal cells are infected with a retrovirus containing the transgene whereby the germ cells of the embryo have the transgene chromosomally integrated therein. When the animals to be made transgenic are avian, because avian fertilized ova generally go through cell division for the first twenty hours in the oviduct, microinjection into the pronucleus of the fertilized egg is problematic due to the inaccessibility of the pronucleus. Therefore, of the methods to make transgenic animals described generally above, retrovirus infection is preferred for avian species, for example as described in U.S. Pat. No. 5,162,215. If microinjection is to be used with avian species, however, a recently published procedure by Love et al., (Biotechnology, Jan. 12, 1994) can be utilized whereby the embryo is obtained from a sacrificed hen approximately two and one-half h after the laying of the previous laid egg, the transgene is microinjected into the cytoplasm of the germinal disc and the embryo is cultured in a host shell until maturity. When the animals to be made transgenic are bovine or porcine, microinjection can be hampered by the opacity of the ova thereby making the nuclei difficult to identify by traditional differential interference-contrast microscopy. To overcome this problem, the ova can first be centrifuged to segregate the pronuclei for better visualization.

The animals of the invention are murine (e.g., mouse). The transgenic mice of the invention are produced by introducing “transgenes” into the germline of the mice. Embryonic target cells at various developmental stages can be used to introduce transgenes. Different methods are used depending on the stage of development of the embryonic target cell. The zygote is the best target for microinjection. The use of zygotes as a target for gene transfer has a major advantage in that in most cases the injected DNA will be incorporated into the host gene before the first cleavage (Brinster et al., Proc. Natl. Acad. Sci. USA 82:4438-4442, 1985). As a consequence, all cells of the transgenic mouse will carry the incorporated transgene. This will in general also be reflected in the efficient transmission of the transgene to offspring of the founder since 50% of the germ cells will harbor the transgene.

The term “transgenic” is used to describe an animal which includes exogenous genetic material within all of its cells. A “transgenic” animal can be produced by cross-breeding two chimeric animals which include exogenous genetic material within cells used in reproduction. Twenty-five percent of the resulting offspring will be transgenic i.e., animals which include the exogenous genetic material within all of their cells in both alleles. 50% of the resulting animals will include the exogenous genetic material within one allele and 25% will include no exogenous genetic material.

In the microinjection method useful in the practice of the subject invention, the transgene is digested and purified free from any vector DNA e.g. by gel electrophoresis. It is preferred that the transgene include an operatively associated promoter which interacts with cellular proteins involved in transcription, ultimately resulting in constitutive expression. Promoters useful in this regard include those from cytomegalovirus (CMV), Moloney leukemia virus (MLV), and herpes virus, as well as those from the genes encoding metallothionin, skeletal actin, P-enolpyruvate carboxylase (PEPCK), phosphoglycerate (PGK), DHFR, and thymidine kinase. Promoters for viral long terminal repeats (LTRS) such as Rous Sarcoma Virus can also be employed. Constructs useful in plasmid transfection of embryonic stem cells will employ additional regulatory elements well known in the art such as enhancer elements to stimulate transcription, splice acceptors, termination and polyadenylation signals, and ribosome binding sites to permit translation.

Retroviral infection can also be used to introduce transgene into a mice. The developing mouse embryo can be cultured in vitro to the blastocyst stage. During this time, the blastomeres can be targets for retro viral infection (Jaenich, R., Proc. Natl. Acad. Sci USA 73:1260-1264, 1976). Efficient infection of the blastomeres is obtained by enzymatic treatment to remove the zona pellucida (Hogan, et al. (1986) in Manipulating the Mouse Embryo, Cold Spring Harbor Laboratory Press, Cold Spring Harbor, N.Y.). The viral vector system used to introduce the transgene is typically a replication-defective retro virus carrying the transgene (Jahner, et al., Proc. Natl. Acad. Sci. USA 82:6927-6931, 1985; Van der Putten, et al., Proc. Natl. Acad. Sci USA 82:6148-6152, 1985). Transfection is easily and efficiently obtained by culturing the blastomeres on a monolayer of virus-producing cells (Van der Putten, supra; Stewart, et al., EMBO J. 6:383-388, 1987). Alternatively, infection can be performed at a later stage. Virus or virus-producing cells can be injected into the blastocoel (D. Jahner et al., Nature 298:623-628, 1982). Most of the founders will be mosaic for the transgene since incorporation occurs only in a subset of the cells which formed the transgenic nonhuman animal. Further, the founder may contain various retro viral insertions of the transgene at different positions in the genome which generally will segregate in the offspring. In addition, it is also possible to introduce transgenes into the germ line, albeit with low efficiency, by intrauterine retroviral infection of the midgestation embryo (D. Jahner et al., supra).

A third type of target cell for transgene introduction is the embryonal stem cell (ES). ES cells are obtained from pre-implantation embryos cultured in vitro and fused with embryos (M. J. Evans et al. Nature 292:154-156, 1981; M. O. Bradley et al., Nature 309: 255-258, 1984; Gossler, et al., Proc. Natl. Acad. Sci USA 83:9065-9069, 1986; and Robertson et al., Nature 322:445-448, 1986). Transgenes can be efficiently introduced into the ES cells by DNA transfection or by retro virus-mediated transduction. Such transformed ES cells can thereafter be combined with blastocysts from a nonhuman animal. The ES cells thereafter colonize the embryo and contribute to the germ line of the resulting chimeric animal. (For review see Jaenisch, R., Science 240: 1468-1474, 1988).

“Transformed” means a cell into which (or into an ancestor of which) has been introduced, by means of recombinant nucleic acid techniques, a heterologous nucleic acid molecule. “Heterologous” refers to a nucleic acid sequence that either originates from another species or is modified from either its original form or the form primarily expressed in the cell.

“Transgene” means any piece of DNA which is inserted by artifice into a cell, and becomes part of the genome of the organism (i.e., either stably integrated or as a stable extrachromosomal element) which develops from that cell. Such a transgene may include a gene which is partly or entirely heterologous (i.e., foreign) to the transgenic organism, or may represent a gene homologous to an endogenous gene of the organism. Included within this definition is a transgene created by the providing of an RNA sequence which is transcribed into DNA and then incorporated into the genome. The transgenes of the invention include DNA sequences that encode Phospholipid Scramblase polypeptide-sense and antisense polynucleotides, which may be expressed in a transgenic non-human animal. The term “transgenic” as used herein additionally includes any organism whose genome has been altered by in vitro manipulation of the early embryo or fertilized egg or by any transgenic technology to induce a specific gene knockout. As used herein, the term “transgenic” includes any transgenic technology familiar to those in the art which can produce an organism carrying an introduced transgene or one in which an endogenous gene has been rendered non-functional or “knocked out”.

In another series of embodiments, the present invention provides methods of inhibiting or preventing viral infection by introducing into viral-infected cells or uninfected cells a Phospholipid Scramblase polypeptide or fragments thereof containing the amino acid sequence PPxY. The N-terminal regions of human and mouse Phospholipid Scramblase 1 share with diverse types of membrane bound viruses late function PPxY motifs required for release of virus particles from cells. The PPxY motifs in Phospholipid Scramblase 1 suppress virus budding by competing with viral M or Gag proteins for binding to cellular WW domain proteins.

As used herein, “viral-infected cells” refers to cells having an infection of a rhabdovirus, a filovirus, a retrovirus, a flavivirus, a coronavirus, a orthomyxovirus, a bunyavirus, a hepadnavirus, a herpesvirus, a poxvirus, a togavirus, a iridovirus, a paramyxovirus or an arenavirus, an infection of a rhabdovirus, a filovirus, a retrovirus, and the like. A virus infection can be an HIV infection, an Ebola virus infection, a Marburg virus infection or a Rabies virus infection. A virus infection can be an infection of a membrane bound virus.

As used herein, “inhibiting” refers to arresting the development of a disease, disorder or condition and/or causing the reduction, remission, or regression of a disease, disorder or condition. As used herein, “preventing” refers to stopping the initiation of a disease or condition. Those of skill in the art will understand that various methodologies and assays may be used to assess the development of a disease, disorder or condition, and similarly, various methodologies and assays may be used to assess the reduction, remission or regression of a disease, disorder or condition.

In an embodiment of the invention the polypeptides and fragments bind to cellular WW domain proteins. In another embodiment of the invention, the Phospholipid Scramblase polypeptide is interferon-inducible. Interferons are a family of pleiotropic cytokines responsible for providing vertebrates with innate immunity against a wide-range of viruses and other microbial pathogens. In addition, interferons have anti-tumor activities due to their anti-proliferative, apoptotic and immunoregulatory properties. Type I interferon (14 subtypes of IFN-α, IFN-β and IFN-ω) are induced in most cell types in response to virus infections whereas type II interferon (IFN-γ) is induced in T lymphocytes and NK cells in response to immune and inflammatory stimulation.

In yet another embodiment of the invention, the method further comprises administering an interferon. As used herein, “administering” refers to means for providing a therapeutically effective amount of a compound to a subject, using oral, sublingual intravenous, subcutaneous, transcutaneous, intramuscular, intracutaneous, intrathecal, epidural, intraoccular, intracranial, inhalation, rectal, vaginal, and the like administration. Administration in the form of creams, lotions, tablets, capsules, pellets, dispersible powders, granules, suppositories, syrups, elixirs, lozenges, injectable solutions, sterile aqueous or non-aqueous solutions, suspensions or emulsions, patches, and the like, is also contemplated. The active ingredients may be compounded with non-toxic, pharmaceutically acceptable carriers including, glucose, lactose, gum acacia, gelatin, mannitol, starch paste, magnesium trisilicate, talc, corn starch, keratin, colloidal silica, potato starch, urea, dextrans, and the like.

The preferred route of administration will vary with the clinical indication. Some variation in dosage will necessarily occur depending upon the condition of the patient being treated, and the physician will, in any event, determine the appropriate dose for the individual patient. The effective amount of compound per unit dose depends, among other things, on the body weight, physiology, and chosen inoculation regimen. A unit dose of compound refers to the weight of compound without the weight of carrier (when carrier is used).

In another embodiment of the invention, a method is provided for identifying a compound that modulates Phospholipid Scramblase polypeptide activity. The method includes incubating the compound with a cell expressing a Phospholipid Scramblase polypeptide under conditions sufficient to permit the compound to interact with the cell and comparing the cellular response in a cell incubated with the compound with the cellular response of a cell not incubated with the compound, thereby identifying a compound that modulates Phospholipid Scramblase polypeptide activity.

The term “modulate” with respect to activity of polypeptide envisions the suppression of Phospholipid Scramblase protein activity or expression when Phospholipid Scramblase protein has an increased activity as compared to a control. The term “modulate” also includes the augmentation of the expression of Phospholipid Scramblase polypeptide when it has a decreased activity as compared to a control.

In another embodiment of the invention, there is provided a method of treating a disorder associated with Phospholipid Scramblase polypeptide activity. The method includes administering to a subject in need thereof a therapeutically effective amount of a compound that modulates a Phospholipid Scramblase polypeptide activity.

In another embodiment of the invention, a method is provided for diagnosis of a subject having or at risk of having a Phospholipid Scramblase-related disorder. A preferred embodiment of the present invention, includes a method for detecting in the subject a level or activity of a Phospholipid Scramblase polypeptide wherein a difference in the level or activity as compared to a normal subject is indicative of a Phospholipid Scramblase-related disorder. In one embodiment of the invention, the level or activity of a Phospholipid Scramblase polypeptide in the subject having or at risk of having Phospholipid Scramblase-related disorder is lower than the level of a Phospholipid Scramblase polypeptide in a normal subject.

In another embodiment, the invention provides a method for diagnosing a subject having or at risk of having a virus infection. Virus infection includes but is not limited to an infection of a rhabdovirus, a filovirus, a retrovirus, a flavivirus, a coronavirus, a orthomyxovirus, a bunyavirus, a hepadnavirus, a herpesvirus, a poxvirus, a togavirus, a iridovirus, a paramyxovirus or an arenavirus, an infection of a rhabdovirus, a filovirus, and a retrovirus. A virus infection can be an HIV infection, an Ebola virus infection, a Marburg virus infection or a Rabies virus infection.

In addition to virus infection, another Phospholipid Scramblase-related disorder is cancer. Cancer includes but is not limited to hairy cell leukemia, chronic myelogenous leukemia, myeloma, melanoma, renal cell carcinoma, Kaposi's sarcoma, follicular lymphoma, thrombocythemia, and erythroleukemia.

The invention also provides a method of increasing or extending the viability of mammalian cells or tissue by inhibiting the expression of a Phospholipid Scramblase polynucleotide within the cell or tissue. As used herein, “increasing the viability” refers to any modification to a cell or tissue that improves the general physical condition and vigor of the cell or tissue. As used herein, “extending the viability” refers to any modification to a cell or tissue that increases the longevity of the cell or tissue.

Yet another aspect of the invention pertains to a method of treating a patient having or at risk of having a disorder associated with a Phospholipid Scramblase polypeptide. The method includes introducing into the patient a polynucleotide encoding the Phospholipid Scramblase polypeptide operatively linked to a regulatory sequence (see Anderson, Nature 392:25-30 (1998)).

One approach for in vivo introduction of nucleic acid encoding one of the subject proteins into a patient is by use of a viral vector containing nucleic acid, e.g. a cDNA, encoding the gene product. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid.

Retrovirus vectors and adeno-associated virus vectors are generally understood to be the recombinant gene delivery system of choice for the transfer of exogenous genes in vivo, particularly into humans. These vectors provide efficient delivery of genes into cells, and the transferred nucleic acids are stably integrated into the chromosomal DNA of the host. A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. (1990) Blood 76:271). Thus, recombinant retrovirus can be constructed in which part of the retroviral coding sequence (gag, pol, env) has been replaced by nucleic acid encoding a Phospholipid Scramblase polypeptide, rendering the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al., (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines for preparing both ecotropic and amphotropic retroviral systems include .psi.Crip, .psi.Cre, .psi.2 and .psi.Am. Retroviruses have been used to introduce a variety of genes into many different cell types, including neural cells, epithelial cells, endothelial cells, lymphocytes, myoblasts, hepatocytes, bone marrow cells, in vitro and/or in vivo (see for example Eglitis, et al., (1985) Science 230:1395-1398; Danos and Mulligan (1988) Proc. Natl. Acad. Sci. USA 85:6460-6464; Wilson et al., (1988) Proc. Natl. Acad. Sci. USA 85:3014-3018; Armentano et al., (1990) Proc. Natl. Acad. Sci. USA 87:6141-6145; Huber et al., (1991) Proc. Natl. Acad. Sci. USA 88:8039-8043; Ferry et al., (1991) Proc. Natl. Acad. Sci. USA 88:8377-8381; Chowdhury et al., (1991) Science 254:1802-1805; van Beusechem et al., (1992) Proc. Natl. Acad. Sci. USA 89:7640-7644; Kay et al., (1992) Human Gene Therapy 3:641-647; Dai et al., (1992) Proc. Natl. Acad. Sci USA 89:10892-10895; Hwu et al., (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

In choosing retroviral vectors as a gene delivery system for the subject Phospholipid Scramblase genes, it is important to note that a prerequisite for the successful infection of target cells by most retroviruses, and therefore of stable introduction of the recombinant Phospholipid Scramblase gene, is that the target cells must be dividing. In general, this requirement will not be a hindrance to use of retroviral vectors to deliver antagonistic Phospholipid Scramblase gene constructs. In fact, such limitation on infection can be beneficial in circumstances wherein the tissue (e.g. nontransformed cells) surrounding the target cells does not undergo extensive cell division and is therefore refractory to infection with retroviral vectors.

Furthermore, it has been shown that it is possible to limit the infection spectrum of retroviruses and consequently of retroviral-based vectors, by modifying the viral packaging proteins on the surface of the viral particle (see, for example PCT publications WO93/25234, WO94/06920, and WO94/11524). For instance, strategies for the modification of the infection spectrum of retroviral vectors include: coupling antibodies specific for cell surface antigens to the viral env protein (Roux et al., (1989) Proc. Natl. Acad. Sci. USA 86:9079-9083; Julan et al., (1992) J. Gen Virol 73:3251-3255; and Goud et al., (1983) Virology 163:251-254); or coupling cell surface ligands to the viral env proteins (Neda et al., (1991) J. Biol Chem. 266:14143-14146). Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, and can also be used to convert an ecotropic vector in to an amphotropic vector.

Moreover, use of retroviral gene delivery can be further enhanced by the use of tissue- or cell-specific transcriptional regulatory sequences which control expression of the Phospholipid Scramblase gene of the retroviral vector.

Another viral gene delivery system useful in the present invention utilizes adenovirus-derived vectors. The genome of an adenovirus can be manipulated such that it encodes a gene product of interest, but is inactivate in terms of its ability to replicate in a normal lytic viral life cycle (see, for example, Berkner et al., (1988) BioTechniques 6:616; Rosenfeld et al., (1991) Science 252:431-434; and Rosenfeld et al., (1992) Cell 68:143-155). Suitable adenoviral vectors derived from the adenovirus strain Ad type 5 dl324 or other strains of adenovirus (e.g., Ad2, Ad3, Ad7 etc.) are well known to those skilled in the art. Recombinant adenoviruses can be advantageous in certain circumstances in that they are not capable of infecting nondividing cells and can be used to infect a wide variety of cell types, including airway epithelium (Rosenfeld et al., (1992) cited supra), endothelial cells (Lemarchand et al., (1992) Proc. Natl. Acad. Sci USA 89:6482-6486), hepatocytes (Herz and Gerard, (1993) Proc. Natl. Acad. Sci. USA 90:2812-2816) and muscle cells (Quantin et al., (1992) Proc. Natl. Acad. Sci USA 89:2581-2584). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham (1986) J. Virol. 57:267). Most replication-defective adenoviral vectors currently in use and therefore favored by the present invention are deleted for all or parts of the viral E1 and E3 genes but retain as much as 80% of the adenoviral genetic material (see, e.g., Jones et al., (1979) Cell 16:683; Berkner et al., supra; and Graham et al., in Methods in Molecular Biology, E. J. Murray, Ed. (Humana, Clifton, N.J., 1991) vol. 7. pp. 109-127). Expression of the inserted Phospholipid Scramblase gene can be under control of, for example, the E1A promoter, the major late promoter (MLP) and associated leader sequences, the E3 promoter, or exogenously added promoter sequences.

Yet another viral vector system useful for delivery of the subject genes is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro. and Immunol. (1992) 158:97-129). It is also one of the few viruses that may integrate its DNA into non-dividing cells, and exhibits a high frequency of stable integration (see for example Flotte et al., (1992) Am. J. Respir. Cell. Mol. Biol. 7:349-356; Samulski et al., (1989) J. Virol. 63:3822-3828; and McLaughlin et al., (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al., (1985) Mol. Cell. Biol. 5:3251-3260 can be used to introduce DNA into cells. A variety of nucleic acids have been introduced into different cell types using AAV vectors (see for example Hermonat et al., (1984) Proc. Natl. Acad. Sci. USA 81:6466-6470; Tratschin et al., (1985) Mol. Cell. Biol. 4:2072-2081; Wondisford et al., (1988) Mol. Endocrinol. 2:32-39; Tratschin et al., (1984) J. Virol. 51:611-619; and Flotte et al., (1993) J. Biol. Chem. 268:3781-3790).

In addition to viral transfer methods, such as those illustrated above, non-viral methods can also be employed to cause expression of a Phospholipid Scramblase polypeptide in the tissue of an animal. Most nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of the subject Phospholipid Scramblase gene by the targeted cell. Exemplary gene delivery systems of this type include liposomal derived systems, poly-lysine conjugates, and artificial viral envelopes.

In clinical settings, the gene delivery systems can be introduced into a patient by any of a number of methods, each of which is familiar in the art. For instance, a pharmaceutical preparation of the gene delivery system can be introduced systemically, e.g. by intravenous injection, and specific transduction of the construct in the target cells occurs predominantly from specificity of transfection provided by the gene delivery vehicle, cell-type or tissue-type expression due to the transcriptional regulatory sequences controlling expression of the gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g. Chen et al., (1994) Proc. Natl. Acad. Sci. USA 91: 3054-3057).

Moreover, the pharmaceutical preparation can consist essentially of the gene delivery system in an acceptable diluent, or can comprise a slow release matrix in which the gene delivery vehicle is imbedded. Alternatively, where the complete gene delivery system can be produced in tact from recombinant cells, e.g. retroviral packages, the pharmaceutical preparation can comprise one or more cells which produce the gene delivery system. In the case of the latter, methods of introducing the viral packaging cells may be provided by, for example, rechargeable or biodegradable devices. Various slow release polymeric devices have been developed and tested in vivo in recent years for the controlled delivery of drugs, including proteinaceous biopharmaceuticals, and can be adapted for release of viral particles through the manipulation of the polymer composition and form. A variety of biocompatible polymers (including hydrogels), including both biodegradable and non-degradable polymers, can be used to form an implant for the sustained release of an the viral particles by cells implanted at a particular target site. Such embodiments of the present invention can be used for the delivery of an exogenously purified virus, which has been incorporated in the polymeric device, or for the delivery of viral particles produced by a cell encapsulated in the polymeric device.

By choice of monomer composition or polymerization technique, the amount of water, porosity and consequent permeability characteristics can be controlled. The selection of the shape, size, polymer, and method for implantation can be determined on an individual basis according to the disorder to be treated and the individual patient response. The generation of such implants is generally known in the art. See, for example, Concise Encyclopedia of Medical & Dental Materials, ed. by David Williams (MIT Press: Cambridge, Mass., 1990); and the Sabel et al., U.S. Pat. No. 4,883,666. In another embodiment of an implant, a source of cells producing a the recombinant virus is encapsulated in implantable hollow fibers. Such fibers can be pre-spun and subsequently loaded with the viral source (Aebischer et al., U.S. Pat. No. 4,892,538; Aebischer et al., U.S. Pat. No. 5,106,627; Hoffman et al., (1990) Expt. Neurobiol. 110:39-44; Jaeger et al., (1990) Prog. Brain Res. 82:41-46; and Aebischer et al., (1991) J. Biomech. Eng. 113:178-183), or can be co-extruded with a polymer which acts to form a polymeric coat about the viral packaging cells (Lim U.S. Pat. No. 4,391,909; Sefton U.S. Pat. No. 4,353,888; Sugamori et al., (1989) Trans. Am. Artif. Intern. Organs 35:791-799; Sefton et al., (1987) Biotechnol. Bioeng. 29:1135-1143; and Aebischer et al., (1991) Biomaterials 12:50-55). Again, manipulation of the polymer can be carried out to provide for optimal release of viral particles.

The following examples are intended to illustrate but not to limit the invention in any manner, shape, or form, either explicitly or implicitly. While they are typical of those that might be used, other procedures, methodologies, or techniques known to those skilled in the art may alternatively be used.

### EXAMPLE 1

The key to understanding how IFNs do what they do is encoded in the IFN regulated genes. To obtain an unbiased and global profile of IFN stimulated and repressed genes, DNA microarray experiments were performed on IFN-α,-β and -γ treated human HT1080 fibrosarcoma cells. In surveying about 6,800 different human genes, greater than 300 genes were found to be regulated by greater than 2-fold by the IFNs. However, only 26 genes were induced by greater than 4-fold by IFN-α. The IFN-regulated genes may be arranged into groups based on the functions or pathways that they mediate. For instance, the anti-viral action of IFNs involves 2-5A synthetase, protein kinase PKR and the Mx proteins. Antigen presentation and processing genes induced by IFNs included MHCI and II, peptide transporter (TAP) and proteasome subunits (LMP) and protease pathways (ubiquitins). Additional protein families regulated by IFNs include chemokines, GTPases, signaling proteins, heat shock proteins, and apoptosis proteins. While the number of regulated genes is large, genes which are induced by all three types of IFNs, and which are the most prominently upregulated, are likely to have a role in IFN biology. In this regard, the PLSCR1 gene was the thirteenth most highly induced gene, showing 8-, 10-, and 3-fold increases by IFN-α, -β and -γ, respectively. Furthermore, PLSCR1 was only one of two genes among the 17-most highly IFN induced genes that was not previously known to be induced by IFN. This discovery is leading to new ways of thinking about how IFNs affect cell physiology.

### EXAMPLE 2

Analysis of 5′ flanking genomic sequence in reporter constructs showed that transcriptional control of PLSCR1 was entirely regulated by a single IFN-stimulated response element (ISRE) located in the first exon. A similar induction of PLSCR1 by IFN-α2a was also observed in a variety of other human tumor cell lines as well as in human umbilical vein endothelial cells. In these cell lines, the marked increase in PLSCR1 expression by IFN-α2a was not accompanied by increased cell surface exposure of PS. These results suggest that remodeling of the cell surface requires both exposure to IFN and a second yet-to-be identified event to stimulate plasma membrane PL scramblase activity and to mobilize PS to the cell surface. We have recently confirmed that the IFN-inducibility observed for hu PLSCR1 is also shared by the murine orthologue, mu PLSCR1. Experiments to identify the ISRE(s) responsible for IFN-regulated expression of mu PLSCR1 gene (as described for hu PLSCR1) are now in progress.

### EXAMPLE 3

To directly measure the effect of PLSCR1 induction on tumor growth in the absence of other IFN induced proteins, we constitutively expressed hu PLSCR1 cDNA in the human ovarian cancer cell line, HEY1B. The hu PLSCR1 cDNA was subcloned under the control of a CMV promoter in plasmid vector, pcDNAneo3. Transfection of the HEY1B cells was followed by selection in media containing G418. Western blots probed with a monoclonal antibody to PLSCR1 (mab 4D2) revealed that only one of fifty clones expressed high levels of PLSCR1. The clone, S48, expressed about 4-fold more PLSCR1 than the parental cells. This compares with the 10-fold increase in PLSCR1 levels obtained with IFN-α treatment of the cells. The in vitro growth rates of the S48 cells and of the clonal cell line (V24) containing the empty vector was determined in the presence and absence of 1,000 units per ml of IFN-α2a . While the growth of both cell lines was modestly suppressed by IFN, there was no difference in the growth rates of the V24 and S48 cells. In sharp contrast, there was a dramatic difference in the ability of the two cell lines to form tumors after being implanted into nude mice. In these experiments, the V24 cells and the PLSCR1 expressing clone, S48, (10⁶ cells/site) were injected subcutaneously (s.c.) into the flanks of groups of six nude mice. Tumor growth was monitored every 3-4 days with a caliper and the excised tumors were measured upon termination of the experiment. The tumor growth rate of the empty-vector control clone was about 8-fold higher than that of the PLSCR1 (S48) clone. To rule out clonal variation as the cause of the differences, we cloned hu PLSCR1 cDNA into vector pIREShyg (Clontech) which expresses a bicistronic mRNA under the control of a CMV promoter. The first open reading frame is PLSCR1 followed by an internal ribosome entry site (IRES) and a hygromycin B phosphotransferase sequence. Therefore, after transfection of the HEY1B cells and selection in hygromycin-containing media, expression of PLSCR1 was tightly coupled to hygromycin resistance. The result was high expression (about 4- to 10-fold over basal levels) of PLSCR1 in every clone analyzed and as well as in the pool of selected cells (data not shown). The pools of PLSCR1 expressing cells and the empty-vector pool of cells were inoculated s.c. into the flanks of groups of nude mice. The tumor results obtained from the uncloned pooled cells (transfected with PLSCR1 cDNA) were similar to those obtained for the S48 clone, thus eliminating a clonal artefact as the basis for the anti-tumor activity of PLSCR1 (data not shown). In this proposal we will investigate the molecular and cellular mechanism of the anti-tumor effect of PLSCR1.

### EXAMPLE 4

To determine the possible antiviral function of PLSCR1, cell viability and viral yield assays with VSV were performed in the presence or absence of IFN-β on cells expressing hu PLSCR1 cDNA. VSV is a rhabdovirus containing a negative RNA genome with five genes in the order 3′-N-P-M-G-L-5′. Its virions are composed of two main parts, a nucleocapsid or ribonucleoprotein (RNP) core and a lipid bilayer envelope. VSV was chosen for these studies because IFN inhibits its replication at a late stage and it is a membrane-bound virus with a PPxY viral budding motif in its M protein. Hu PLSCR1 is an IFN-induced membrane protein that contains two PPxY motifs near its cytoplasmic N-terminus. Therefore, PLSCR1 could interfere with VSV budding or assembly by competing with VSV M protein for binding to cellular WW domain proteins required for these late viral processes.

The effect of VSV (New Jersey strain) on cell viability was determined in the pool of stably-transfected HEY1B cells expressing PLSCR1 from pIREShyg and the pool of cells stably transfected with empty-vector. Cell protection was measured using the MTS tetrazolium compound (Owen's reagent), a colorimetric indicator of cell metabolism (Promega). Infections were at different multiplicities of infection (MOIs)=0.1, 1.0 and 10 plaque forming units (pfu) per cell for 48 h and results were averaged from five identical wells per treatment. At low MOI's (0.1 and 1.0), cell viability was 53 and 42% in the vector control cells and 76 and 56% in the PLSCR1 expressing cells. At an MOI of 10, the viability of the PLSCR1 cDNA expressing cells was 33% while that of vector control cells was only 7%. Therefore, expression of PLSCR1 resulted in a significant reduction in the cytopathic effect of VSV infection.

To directly determine the effect of PLSCR1 on VSV replication, viral yield assays were performed in the presence or absence of 16 h pretreatments with 20 or 100 units per ml of IFN-β. In the absence of prior IFN treatment, virus yield was suppressed by 2.5-fold in the PLSCR1 expressing cells compared to the empty-vector control cells. Furthermore, overexpression of PLSCR1 resulted in a potent, 25-fold enhancement in the anti-VSV effect of IFN. IFN is known to inhibit VSV at different stages in its life cycle by different anti-viral pathways. Our results indicate that PLSCR1 cooperates with other IFN-induced proteins in the inhibition of VSV replication and PLSCR1 can therefore interfere with VSV assembly or budding by competing with VSV M protein for binding to cellular WW domain proteins.

### EXAMPLE 5

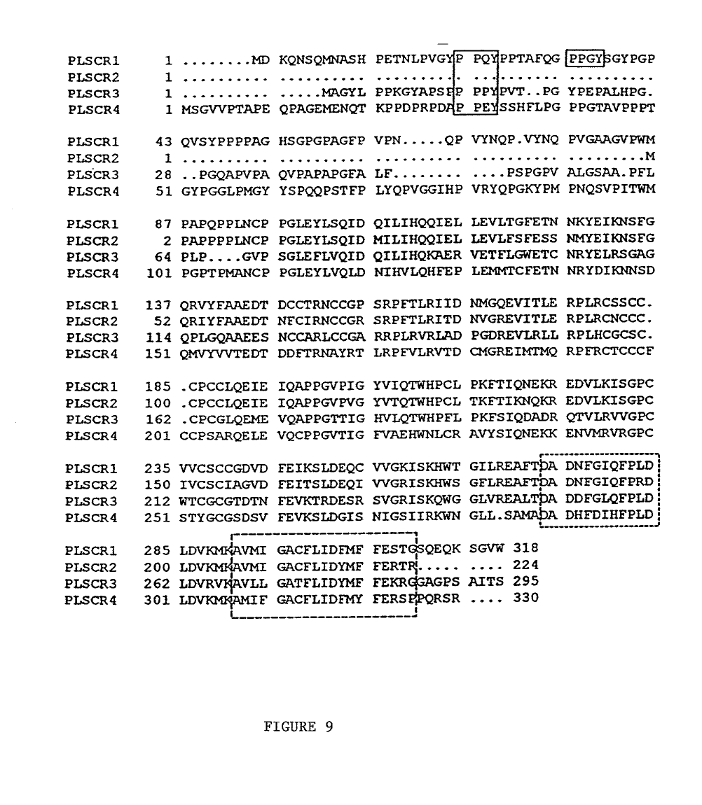

BLAST analysis of the GenBank EST database using hu PLSCR1 cDNA revealed three different families of EST clones that were similar, but distinctly different from the sequence we originally reported for hu PL scramblase (PLSCR1). In order to obtain cDNAs for these putative homologues of huPLSCR1, the relevant EST clones were used to design PCR primers. Full length cDNAs were obtained by PCR using cDNA from multiple human tissues as templates. As illustrated, the cloned cDNAs encode three novel proteins with high homology to huPLSCR1 (FIG. 9). The predicted open reading frames of these putative homologues show sequence identities to huPLSCR1 of 59% (huPLSCR2; 224 AA; GenBank AF159441), 47% (huPLSCR3; 295 AA; GenBank AF159442) and 46% (huPLSCR4; 329 AA; GenBank AF199023), respectively. Corresponding cDNAs of putative murine orthologues of each of the four hu PLSCR family members have also been identified, and similar proteins of unknown function are also predicted in the C. elegans, Drosophila, and porcine genome (data not shown).

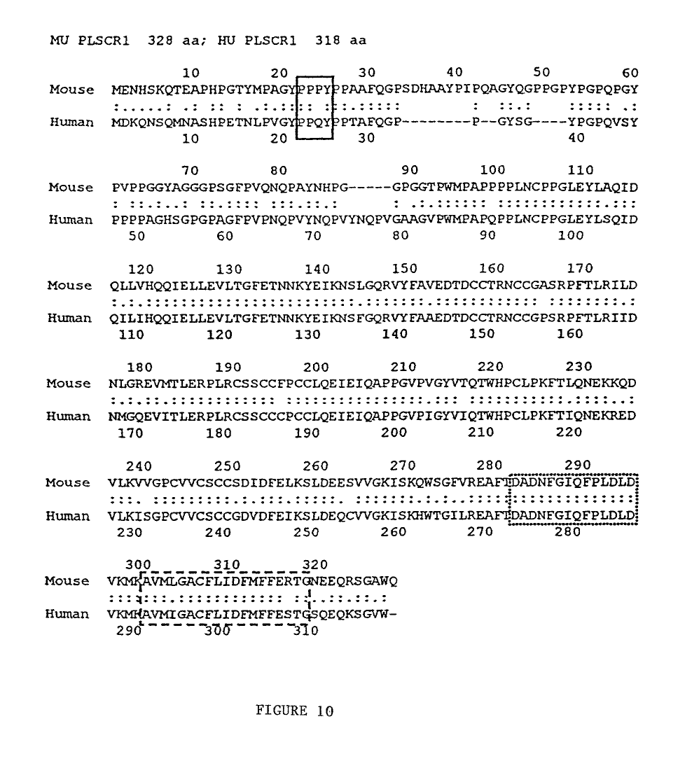

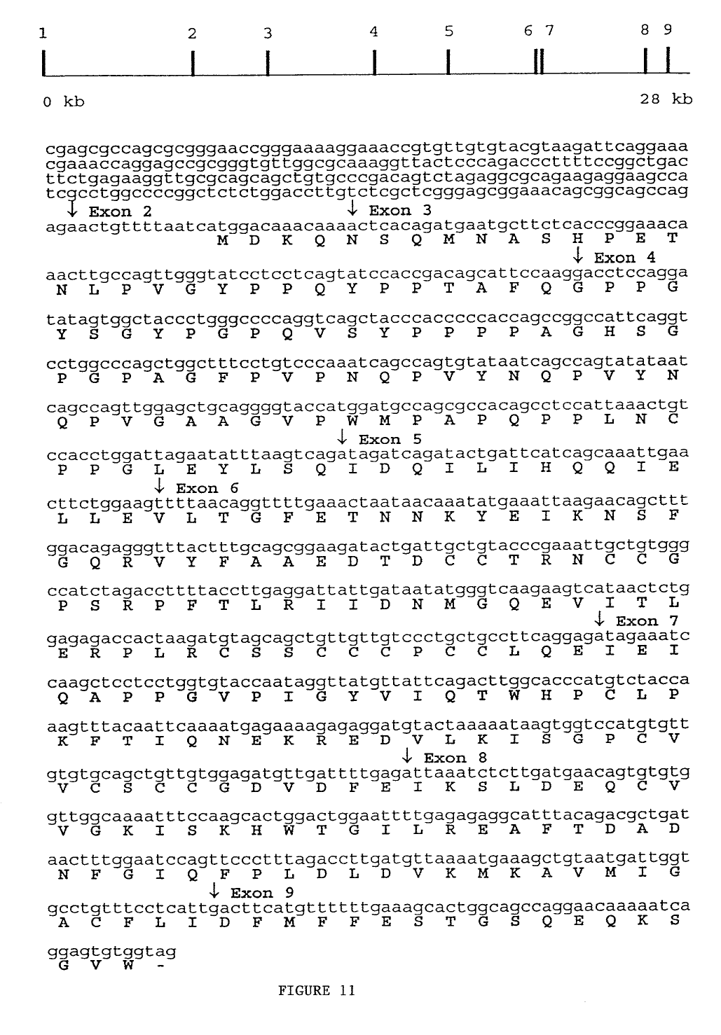

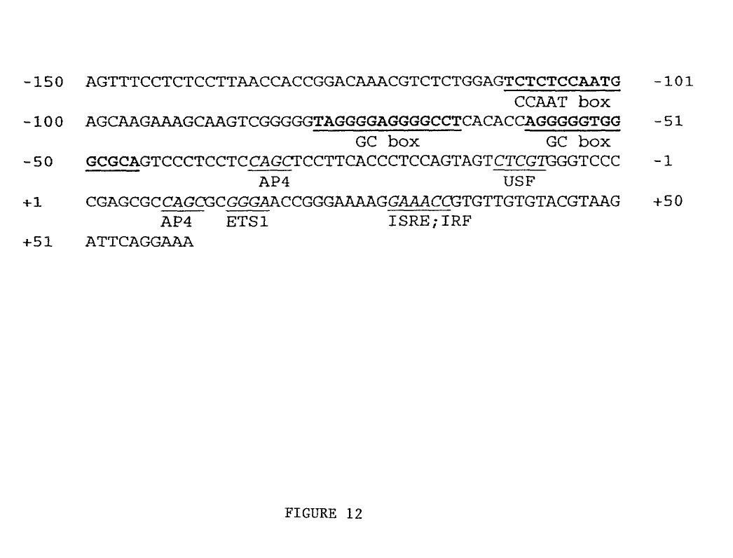

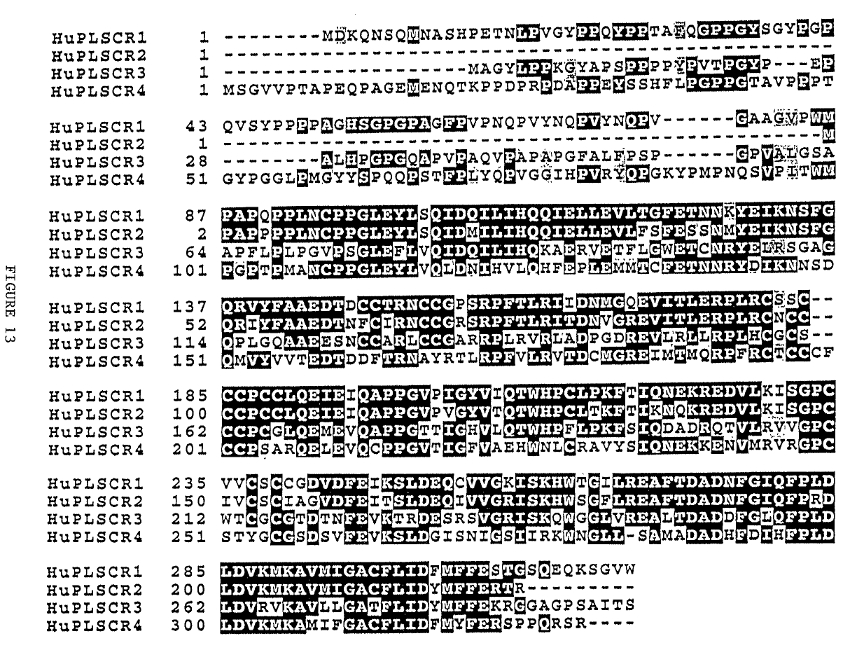

Inspection of the four human PLSCR homologues reveals a low degree of similarity for the proline-rich N-terminal portion of the proteins (AA 1 to 85 in huPLSCR1), and highest degree of identity towards the C-terminus. This includes a highly conserved segment (AA 273 to 284 in huPLSCR1) which has been shown in huPLSCR1 to contain the Ca²⁺-binding site. The sequence of huPLSCR1 predicts a type II membrane protein with single transmembrane domain near the C-terminus (AA 291-309), and most of the polypeptide (AA 1-290) extending into the cytosol. By contrast, such predictions are ambiguous for the newly described homologues, as are predictions of putative intracellular localization. Of note, the predicted open reading frame for huPLSCR2, the closest homologue to huPLSCR1, is missing the proline-rich N-terminus that is characteristic for all other members of this family. As was discussed, this segment in PLSCR1 that is missing in PLSCR2 contains PPxY and PxxP motifs which may serve as binding sites for proteins containing WW or SH3 domains, respectively, and potentially confer on PLSCR1 anti-viral activity. The PPxY and PxxP motifs common to hu PLSCR1, PLSCR3, & PLSCR4 are also conserved in the corresponding mouse orthologue (FIG. 10) as well as in the potential porcine (GENBank F14810) and C. elegans (GENBank Z82084,AF078785) orthologues of PLSCR1. Although binding partners for any of the PLSCR proteins have not been identified to date, it is interesting to note that the functional implication of the missing N-terminal segment in huPLSCR2 may also be a potential loss of interaction with an adaptor or signaling molecule. A similar truncation deleting the proline-rich segment of mu PLSCR1 has previously been associated with leukemogenesis.

Chromosomal Assignment of PL Scramblase Family Members.

Chromosomal localization by analysis of STS sequences of the NCBI Human Gene Map'99 and/or radiation panel hybrid mapping revealed that the genes for hu PLSCR1, PLSCR2, and PLSCR4 are tightly clustered between markers D3S1557 and D3S1306 (164.6-168.3 cM) on chromosome 3 (3q23) at the physical position 537.09 cR₃₀₀₀ (P1.30). HuPLSCR1 was also mapped to chromosome 3 at 3q23 by fluorescence in situ hybridization. By contrast, PLSCR3 maps to chromosome 17 (p13.1) between markers D17S1828 and D17S786 (9.8-18.1 cM) at the physical position 53.50 cR₃₀₀₀ (PO.90).

Tissue Distribution of PL Scramblase Family Members.

Initial insight into the tissue distribution of the four newly-identified members of the PLSCR gene family was obtained by Northern blotting with ³²P-labeled probes specific for huPLSCR1 to huPLSCR4, respectively. Transcripts for PLSCR1 (˜2400 bp and ˜1600 bp) were expressed in spleen, thymus, prostate, testis, uterus, small intestine, colon, peripheral blood lymphocytes, heart, placenta, lung, liver, kidney and pancreas, but below the limits of detection in brain and skeletal muscle. By contrast, PLSCR2 (˜1600 bp) was detected only in testis. PLSCR3 (˜2400 bp and ˜1600 bp ) was detected in spleen, thymus, prostate, uterus, small intestine, colon, PBL, skeletal muscle, heart, placenta, lung, kidney and pancreas, but not in testis, brain or liver. PLSCR4 (˜3600 bp) was detected in all tissues examined except peripheral blood lymphocytes, and was the only PLSCR family member detected in brain.

Antibody Probes of PLSCR Family Members.

We have several monoclonal antibodies specific for hu PLSCR1 that do not cross-react with hu PLSCR2-4. Two of the mabs have been found to cross-react with mu PLSCR1 and can be used to selectively monitor expression of the protein in murine cells and tissue. In order to develop additional antibody probes, peptides corresponding to unique sequence identified in the various PLSCR homologues have been synthesized and peptide-KLH conjugates injected into rabbits for antisera production.

These antisera were analyzed by ELISA and Western blotting against recombinant PLSCR1-4 produced in E. coli as MBP-fusion proteins. At the present time, we have available high titer antisera selective for hu and mu PLSCR1-3. Immunizations with peptides derived from hu and mu PLSCR4 are now in progress. Thus, we anticipate that antibody reagents suitable for monitoring selective protein expression of each of the four PLSCR family members in both human and mouse cells and tissues will be available prior to start of YEAR 1 of the proposed Project.

### EXAMPLE 6

Under contract between Scripps/BCSEW and Lexicon Genetics, Inc., we initiated genomic cloning and Cre-Lox targeted disruption of the murine PLSCR gene locus in 1998. This was prior to our discovery that PLSCR is a multigene family of proteins in both mouse and man, currently shown to include four expressed genes. We now recognize that the original mouse orthologue of human PL scramblase that we had cloned and targeted for gene disruption is mu PLSCR2, not the true orthologue of hu PLSCR1. We therefore recently cloned and targeted disruption of the mu PLSCR1 gene (see FIG. 10). In both cases (PLSCR1 & PLSCR2), the targeting construct was designed to disrupt exon 8, which (by sequence alignment to hu PLSCR1) is predicted to contain the Ca²⁺ binding and putative transmembrane segments of the proteins. The PLSCR2 knockout was completed last year and the breeding colonies transferred to Scripps for use in this Project. To date, no abnormality has been identified in the homozygote PLSCR2^{-/-} animals. As of date of submission of this application (Jan. 29, 2000) matings of the chimeric mice containing the PLSCR1 gene disruption were performed, and 14 resulting agouti pups are now being analyzed for germline transmission. We anticipate that breeding colonies will be transferred to Scripps Transgenic facility in the Spring of '00 for use in this Project. No information is now available on the viability or phenotype of the homozygous PLSCR1^{-/-} animals.

### EXAMPLE 7

Cloning of Human PL Scramblase 1 Gene.

A BAC-human genomic library (Genome System Inc., St. Louis) was screened with a 1.445 kb HuPLSCR1 cDNA probe (GenBank accession number AF098642) by hybridization. A positive clone of approximately 100 kb was obtained, digested with EcoRI, and the fragments were cloned into pcDNA3 (Invitrogen). Subclones were identified by hybridization with digoxigenin-labeled HuPLSCR1 cDNA probe, and DNA inserts were sequenced on an ABI DNA Sequencer Model 373 Stretch (Applied Biosystems) using PRISM Ready Reaction DyeDeoxy Terminator Cycle Sequencing Kit (Perkin Elmer).

Construction of 5′ Flanking Region Deletions of PLSCR1 Gene.

A 4180 bp DNA fragment consisting of the 5′flanking region (0 to -4120) and the first 60 bp of the first exon (+1 to +60) of the HuPLSCR1 gene was cloned into pGL3-basic-luciferase reporter vector (Promega). In order to identify the promoter region of the gene, the 5′ flanking DNA was serially deleted both from the 5′ and the 3′ end by PCR-mediated deletion and cloned into pGL3-basic-luciferase reporter vector for analysis.

Cell Culture and Transfection of Daudi Cells.

The Burkitt's B cell lymphoma cell line Daudi was cultured in RPMI 1640 complete medium with 20% fetal bovine serum, 100 U of penicillin/ml, and 100 μg of streptomycin/ml, at 37° C., 5% CO₂. Cells were washed and suspended to 1.35×10⁷/ml in OPTI-MEM. To 0.8 ml of cell suspension in a 0.4 cm electroporation cuvette, 20 μg of pGL3-5′flanking region or deletions of HuPLSCR1 and 20 μg of pSV-β-galactosidase (Promega) were added. The mixture was incubated for 10 min on ice, and electroporated at 380 V and 500 μF using a Bio-Rad Gene Pulser II (Bio-Rad). After incubation for 10 min at 37° C. the cells were plated in 10 ml of RPMI complete medium. Forty-eight hours later, transfected cells were harvested for luciferase and β-galactosidase assay.

Luciferase and β-galactosidase Assay.

Luciferase activity was measured with a Luciferase Assay Kit (Promega). In brief, transfected Daudi cells were harvested, washed with PBS and lysed for 15 min with Reporter lysis buffer. The cell lysate was vortexed for 15 seconds and centrifuged at 12,000× g at 4° C. for 2 min. In a 96 well plate, 20 μl of lysate was mixed with 100 μl of luciferase Assay buffer by automated reagent injection using a MicroLumatPlus microplate luminometer (EG&G Berthold), and luminescence was measured for 30 seconds. β-Galactosidase activity was determined with o-nitrophenyl-β-D-galactopyranoside as substrate. 100 μl of cell lysate was incubated with 100 μl of 4.4 mM o-nitrophenyl-β-D-galactopyranoside for lh at 37° C., and absorbance was read at 420 nm. Luciferase activity was expressed in arbitrary light units, and corrected for transfection efficiency of β-galactosidase.

Cloning of PL Scramblase Family Members.

Blast search of the GenBank database of expressed sequence tags (EST) with HuPLSCR1 cDNA identified three distinct clusters of EST clones each displaying overlapping identities. Appropriate EST clones were obtained from American Type Culture Collection, sequenced, and the information was used to design PCR primers specific for the 3′ and 5′ ends of the various homologues. Full length cDNAs were obtained by PCR using a human erythroleukemia cell (HEL) cDNA library (Clontech; for HuPLSCR2 and HuPLSCR3) or human multiple tissue cDNA (Clontech; pancreas; for HuPLSCR4) as template. Each PCR reaction and cloning was performed in triplicates, and Advantage HP₂ DNA polymerase mix (Clontech) was used to decrease PCR-mediated error. PCR products were cloned into pCR2.1 (Invitrogen) for sequencing.

Chromosomal Mapping.

The chromosomal location for HuPLSCR2 was determined using the GeneBridge 3 Human Radiation hybrid panel and oligonucleotides 5′-CCTGGTGCTTAGGGTAGACAATATG-3′ and 5′-CTGACGTCCTGGGTAGAAGGCCTGGG-3′ as the forward and reverse primers, respectively (Research Genetics, Huntsville, Ala.). The primers flank a small intron (88 bp) within the 5′ untranslated region of HuPLSCR2, giving a PCR product of 314 base pairs. The map position was calculated using the Stanford server (http://www-shgc.stanford.edu).

Tissue Distribution.

Human multi-tissue Northern blots (Clontech) were hybridized to random prime labeled cDNA probes of each HuPLSCR family member. The HuPLSCR1 probe consisted of the 5′ 498 bp of HuPLSCR1 (gbAF098642). The HuPLSCR2 probe (1265 bp) was prepared by digesting EST clone AA813518 with Not1 and Xho1, and the HuPLSCR4 probe (851 bp) by digesting EST clone N78598 with Not1 and Xho1. The cDNA fragments were separated from vector sequences by agarose gel electrophoresis and purifed using Wizard columns (Promega). The cDNA probes were labeled with α-⁼P-dATP (50 μCi/25 ng cDNA, 3000 Ci/mmol; ICN) using the random prime labeling kit from Boehringer Mannheim to a specific activity ≧1×10⁹ dpm/μg. Due to non-specific hybridization of the cDNA probe, an RNA antisense probe was designed for HuPLSCR3. A PCR product of the 3′ untranslated region of HuPLSCR3 was prepared using the forward primer 5′-TGTGAGGAGACCATCACCTCGAC-3′ and reverse primer 5′-AAAGCTGATATGCCTGTGTGCC-3′. The reverse primer contained the T7 promoter sequence (5′-AATTTAATACGACTCACTATAGGG-3′) at the 5′ end. The PCR product was purified using the Qiaquick PCR purification kit (Qiagen). A ³²P-labeled antisense RNA probe was prepared using 50 ng of the PCR product as template in T7 transcription reaction with α-³²P-UTP (800 Ci/mmol; 20 μCi/μl: Amersham) following the instructions included in the T7 Strip-EZ RNA kit (Ambion). Multi-tissue Northern blots were prehybridized for 1 hour at 68° C. in ExpressHyb hybridization buffer (Clontech) followed by hybridization for 18 hours at 68° C. in the same buffer containing 2×10⁶ cpm/ml denatured random prime-labeled probe. For HuPLSCR3, the blots were prehybridized in Ultrahyb hybridization buffer (Ambion) with 100 μg/ml denatured salmon sperm DNA and 50 μg/ml yeast RNA and hybridized in the same buffer containing ³²P-labeled antisense RNA probe (2×10⁶ cpm/ml) at 68° C. for 18 hours. The blots were washed at a final stringency of 0.1× SSC in 0.1% SDS at 50° C. (68° C. for HuPLSCR3), and exposed to Amersham Hyperfilm MP.

### EXAMPLE 8

Human PLSCR1 Gene Structure.

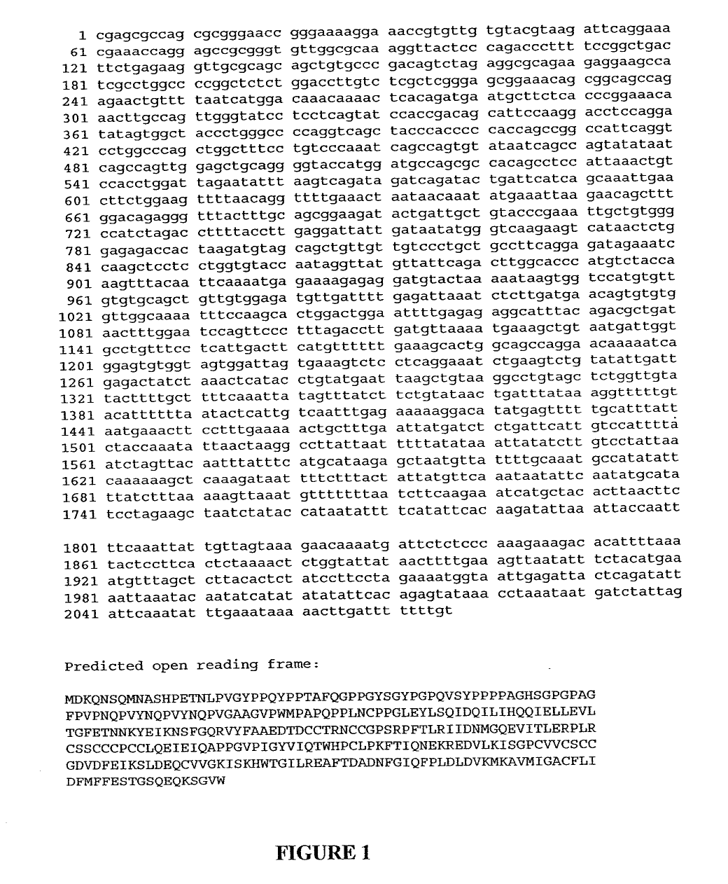

In order to gain insight into the gene organization of HuPLSCR1, a clone of approximately 100 kb of genomic DNA was obtained from a BAC-human genomic library by screening with a HuPLSCR1 Cdna probe. EcoR1 digested fragments were cloned into pcDNA3, and sequence from six different clones was used to deduce approximately 30 kb of HuPLSCR1 genomic DNA. The organization of the gene was deduced by alignment of the genomic sequence with Cdna sequence for HuPLSCR1 (GenBank accession number AF098642). The HuPLSCR1 gene consists of 9 exons, 8 introns and 5′ flanking sequence (deposited under GenBank accession numbers AF153715 and AF224492). As shown in Table 1, invariant gt and ag were found at the intron splice donor and acceptor sites. As illustrated in FIG. 1, the first exon is untranslated, with the open reading frame starting in exon 2. Of interest, Kasukabe et al. (16) reported the occurrence of a truncated form of MuPLSCR1 (termed MmTRA1a), the closest murine orthologue of HuPLSCR1, in a mouse monocytic cell line which was highly leukemogenic when injected into syngeneic or athymic mice. In addition, non-leukemogenic sublines became leukemogenic when transfected with MmTRA1a. By contrast, normal macrophages expressed only full length MuPLSCR1. Comparison of the sequence of MmTRA1a with FIG. 1 reveals that murine MmTRA1a is likely a product of alternative splicing, as the predicted open reading frame reported by Kasukabe et al.(16) starts at a position corresponding to the beginning of Exon 6 in HuPLSCR1. It remains to be determined whether the analogous alternatively spliced forms of HuPLSCR1 are similarly associated with leukemias in man.

### EXAMPLE 9

Promoter Analysis.

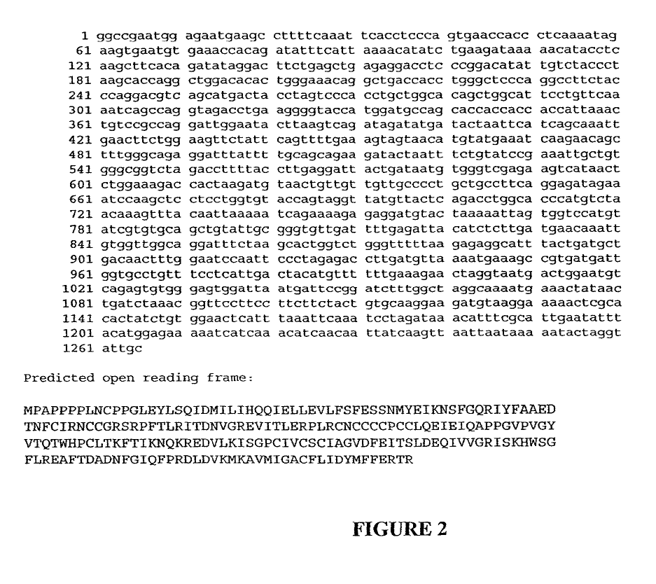

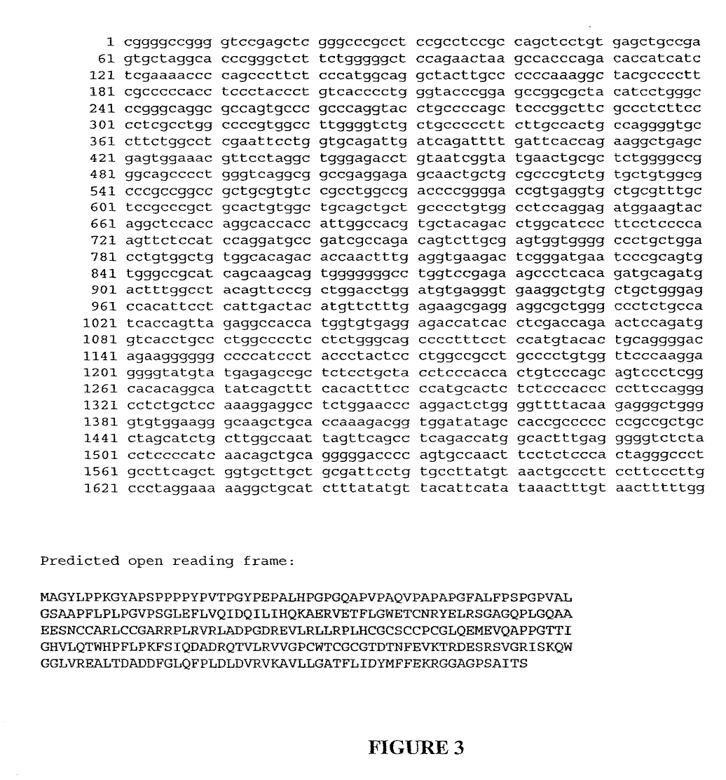

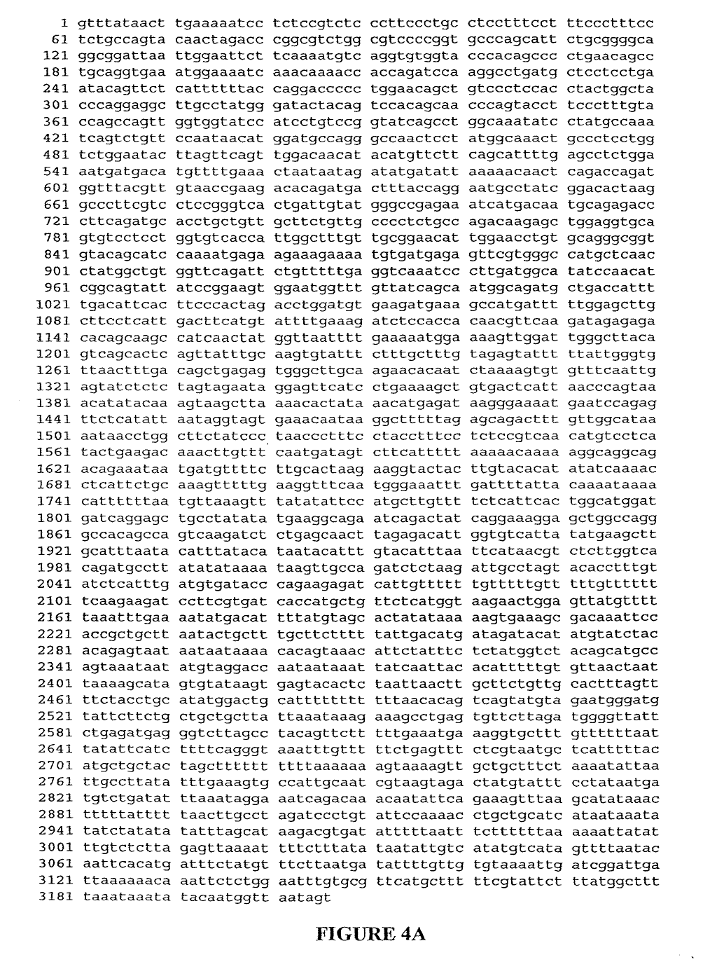

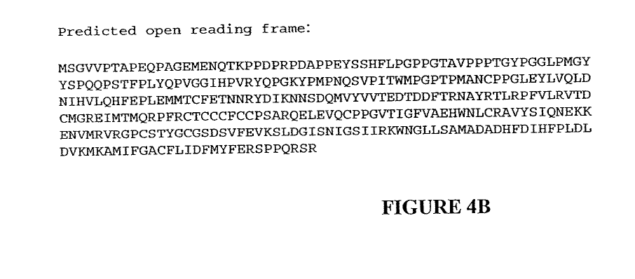

In order to identify the promoter region for HuPLSCR1, luciferase reporter constructs of 5′ flanking sequence and serial 5′ or 3′ deletions were expressed in Daudi cells. As illustrated by the data in FIGS. 2 and 3, a reporter construct containing 5′ untranslated sequence comprised of −4120 to +60 exhibited strong promoter activity. Deletion of sequence from the 5′ end from −4120 bp to −557 bp did not affect promoter activity (FIG. 2). However, deletion from −95 bp of 5′ flanking sequence to +60 bp of the first (untranslated) exon resulted in the loss of more than 97% of promoter activity, locating the promoter of HuPLSCR1 to that region (FIG. 3). Computer analysis of HuPLSCR1 5′ flanking sequence using the MatInspector V2.2 program http://www.gsf.de/biodv/matinspector.html ) revealed two GC boxes (TAGGGGAGGGGCCT at −79 bp to −66 bp, and AGGAGGTGGGCGCA at −59 bp to −46 bp) and a CCAAT box (TCTCTCCAATG at −111 bp to −101 bp) (FIG. 4), consistent with the data in FIG. 3 locating promoter activity to that region. In addition, potential binding sites for transcriptional activators, including activator protein 4 (AP4, upstream stimulating factor (USF), eurkaryotic transcriptional regulator 1 (ETS1), interferon-stimulated response element (ISRE), and interferon regulatory factor (IRF), were identified . We had previously identified the single ISRE that is located in the first untranslated exon (+21 to +35) as the primary site responsible for the upregulation of HuPLSCR1 by interferon-α.

### EXAMPLE 10

Identity of a Novel PL Scramblase Gene Family.

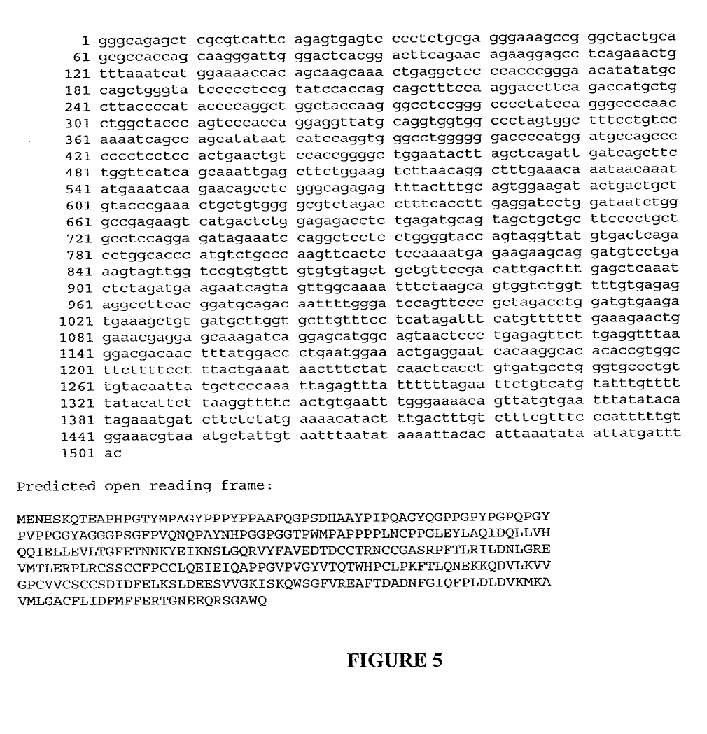

Upon performing BLAST searches of the GenBank EST database with human PL scramblase 1 (HuPLSCR1), we noted three distinct clusters of EST clones that were similar, but distinctly different from the sequence we had originally reported for HuPLSCR1. In order to obtain cDNAs for these putative homologues of HuPLSCR1, sequence derived from relevant EST clones was used to design PCR primers. Full length cDNAs were obtained by PCR using a cDNA library from human erythroleukemia cells (HEL), and cDNA from multiple human tissues as template. As illustrated in FIG. 5, the cloned cDNAs encode three novel proteins with high homology to HuPLSCR1. The predicted open reading frames encode proteins with 59% (HuPLSCR2; 224 AA; GenBank AF159441), 47% (HuPLSCR3; 295 AA; GenBank AF159442) and 46% (HuPLSCR4; 329 AA; GenBank AF199023) identity, respectively, to HuPLSCR1. Furthermore, cDNAs of novel murine orthologues of HuPLSCR3 (MuPLSCR3; 327 AA; GenBank AF159850) and HuPLSCR4 (MuPLSCR4; partial, in progress) have been cloned and sequenced (data not shown). Closer inspection of the four human PLSCR homologues reveals low degree of similarity for the proline-rich aminoterminal portion of the proteins (amino acids 1 to 85 in HuPLSCR1), and highest degree of identity towards the carboxyterminus, including a region (AA 273 to 284 in HuPLSCR1) which has been shown for huPLSCR1 to contain a Ca²⁺-binding site required for the Ca²⁺-induced transmembrane movement of phospholipids. We have previously noted that computer analysis of HuPLSCR1 predicts a type II protein with a transmembrane domain near the carboxyterminus (AA 291-309), and most of the polypeptide (AA 1-290) extending into the cytosol. By contrast, such predictions are ambiguous for the newly described homologues, as are predictions of putative intracellular localization. Of note, the predicted open reading frame for HuPLSCR2, the closest homologue to HuPLSCR1, is missing the proline-rich aminoterminus that is characteristic for all other members of this family. As pointed out previously, this region also contains a number of PXXP motifs which may serve as potential binding sites for proteins containing SH3 domains. In addition, HuPLSCR1, 3, and 4 all contain one or more PPXY motifs, suggesting a potential interaction with proteins containing WW domains. Such domains are primarily found in proteins with signaling or regulatory function. Although binding partners for any of the PLSCR proteins have not been identified to date, it is interesting to note that the functional implication of the missing aminoterminal segment in HPLSCR2 may be a potential loss of interaction with an adaptor or signaling molecule. A similar truncation has previously been noted to confer leukemogenic potential to MuPLSCR1 (MmTRA1a).

### EXAMPLE 11

Chromosomal Assignment of PL Scramblase Family Members.

The chromosomal locations of HuPLSCR1, HuPLSCR3 and HuPLSCR4 were determined from nucleotide sequence homologies to STS sequences found on the NCBI Human Gene Map'99 (http:www.ncbi.nlm.nih.gov/genemap/). The genes for HuPLSCR1(stSG10277) and HuPLSCR4 (gb N78598/G37067) are clustered between markers D3S1557 and D3S1306 (164.6-168.3 Cm) on chromosome 3 (3q23) at the physical position 537.09 Cr₃₀₀₀. HuPLSCR1 has also been independently mapped to chromosome 3 at 3q23 by fluorescence in situ hybridization. A partial sequence for the HuPLSCR3 gene is located between nucleotide 10501 and 9174 of the gene sequence deposited in GenBank under gb AF097738, which also codes for a non-receptor tyrosine kinase gene (nucleotides 531-9180). The non-receptor tyrosine kinase gene has been localized to chromosome 17p13.1 between markers D17S1828 and D17S786 (9.8-18.1 Cm) at the physical position 53.50 Cr₃₀₀₀, thus localizing HuPLSCR3 to that position. The gene for HuPLSCR2 was mapped as described in Experimental Procedures, and was found to be located on chromosome 3 (3q23), closely clustered with HuPLSCR1 and HuPLSCR4, suggesting that these three homologues arose by gene duplication.

### EXAMPLE 12

Tissue Distribution of PL Scramblase Family Members.

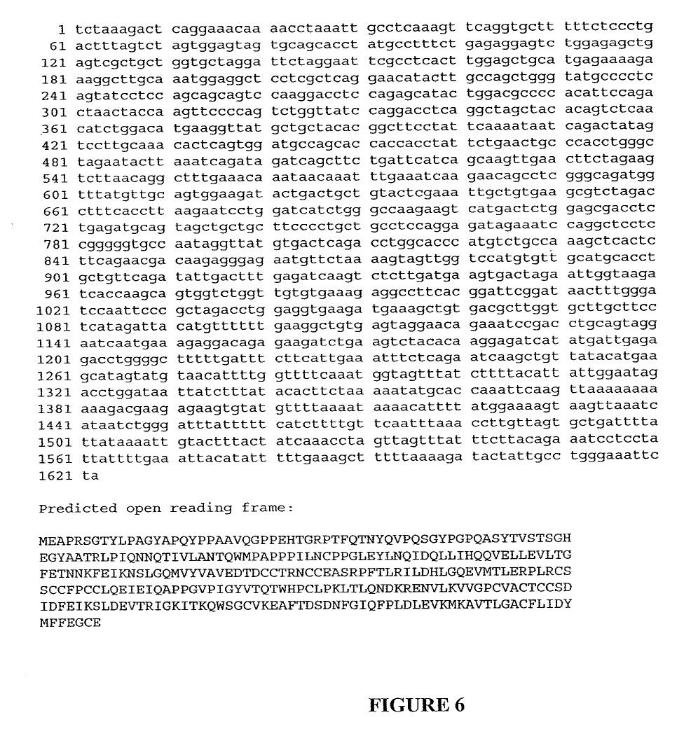

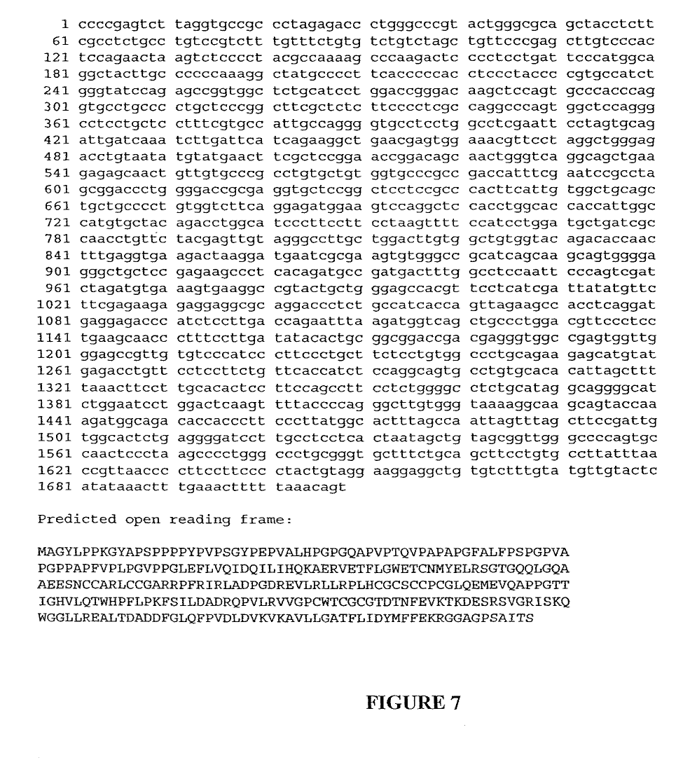

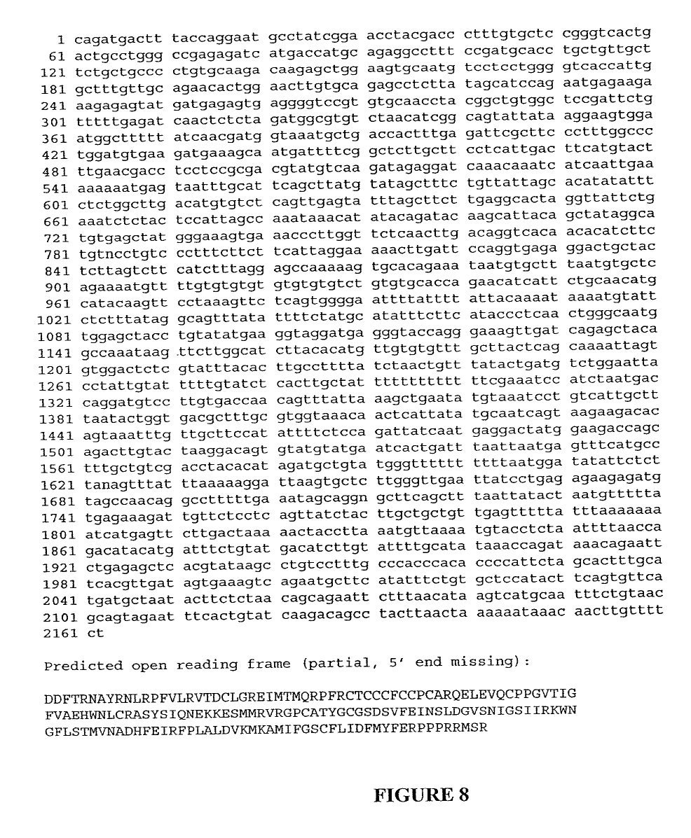

The tissue distribution for the four members of the PL scramblase family of proteins was evaluated by Northern blotting with ³²P-labeled probes specific for HuPLSCR1 to HuPLSCR4, respectively. The specificity of the probes was ascertained by DNA dot blot (FIG. 6). Whereas amounts of mRNA for HuPLSCR2 in many of these tissues were below the limit of detection, the mRNA for the other three homologues were expressed in most of the 16 different tissues examined. However, the expression patterns for these three family members show distinct differences. FIG. 7 shows that mRNA for HuPLSCR1 was below the limits of detection in brain and skeletal muscle. As previously reported, two different size transcripts (˜2.55 kb and 1.6 kb) were detected for HuPLSCR1 in all tissues expressing this gene. Kasukabe et al. have suggested that the different size transcripts arise from alternative polyadenylation signals within the 3′ untranslated region of the HuPLSCR1 gene. Interestingly, the expression of HuPLSCR2 mRNA appears to be highly restricted. Although trace amounts of HuPLSCR2 could be amplified from HEL cells through several rounds of PCR for sequencing purposes (see Experimental Procedures), a 1.6 kb message was only detected in testis. This result was confirmed by probing a human Multi Tissue Expression Array (Clontech, Cat. #7775-1), which again yielded a positive blot against mRNA of testis only. This blot also revealed that in addition to the tissues listed in FIG. 7, HuPLSCR2 message was also not detected in any tissues of the gastrointestinal tract, bladder, ovary, lymph node, bone marrow, and adrenal, thyroid, salivary or mammary gland (results not shown). HuPLSCR3 mRNA was below the limit of detection in testis, brain or liver. Two sizes of mRNA were detected with the HuPLSCR3 specific probe: whereas a 1.8 kb mRNA species was observed for most tissues, a ˜2.1 kb mRNA transcript was detected in skeletal muscle. An mRNA transcript of 4 kb was detected for HuPLSCR4 in all tissues examined except peripheral blood lymphocytes. Importantly, HuPLSCR4 mRNA was the only family member expressed at detectable levels in brain tissue. Whether HuPLSCR1, 3, and 4 have redundant function in a number of tissues, or whether these proteins exhibit activities that are distinct for each family member is the subject of future experimentation.

TABLE 1
Splice donor site Splice acceptor site

---Exon 1---AGCCAGAGgtgcgcgg---Intron T---tttttcagAACTGTTT---Exon 2---
---Exon 2---CAAACAAAgtaagtaa---Intron 2---aattgcagACTCACAG---Exon 3---
---Exon 3---ATTCCAAGgtaaagca---Intron 3---tatttcagGACCTCCA---Exon 4---
---Exon 4---TAAGTCAGgtaatttc---Intron 4---tgctatagATAGATCA---Exon 5---
---Exon 5---TCTGGAAGgtatgtat---Intron 5---gtttttagTTTTAACA---Exon 6---
---Exon 6---TTCAGGAGgtctgtga---Intron 6---ctttgtagATAGAAAT---Exon 7---
---Exon 7---ATTTTGAGgtaagaga---Intron 7---caatttagATTAAATC---Exon 8---
---Exon 8---TCCTCATTgtaagtct---Intron 8---ttatctagGACTTCAT---Exon 9---
Note:
Exon sequence is shown in uppercase, intron sequence in lowercase letters. The invariant gt of intron at splice donor site and ag of intron at splice acceptor site are shown in bold.

### EXAMPLE 13

Materials.

Recombinant human interferon-α2a (IFN-α2a ; 3×10⁶ international units (IU)/ml was from Roche Laboratories (Nutley, N.J.). Bovine prothrombin, factor Va, and factor Xa were obtained from Haematologic Technologies (Essex Junction, Vt.). Chromogenic thrombin substrate CBS 34-47 was from Diagnostica Stago, Asnieres, France). RPMI-1640, Dulbecco's Modified Eagle's Medium (DMEM), Modified Eagle's Medium Essential (MEME), and Opti-MEM were from Gibco-BRL (Grand Island, N.Y.). Murine monoclonal antibody V237 specific for the light chain of factor Va was a gift from Dr. Charles T. Esmon (The Oklahoma Medical Research Fndn, Oklahoma City, Okla.). Murine monoclonal antibody 4D2 was raised against purified recombinant human PLSCR1. Cell lines: Daudi, Raji, HeLa and Jurkat cells were from American Type Culture Collection (Rockville, Md.); human umbilical vein endothelial cells and CS-C medium were from Cell Systems Co. (Kirkland, Wash.); fibrosarcoma cell lines HT1080 and STATI-null U3A cells were a gift of Dr. George R. Stark (Cleveland Clinic Fndn, Cleveland, Ohio).

Cell Culture.

The Burkitt's B cell lymphoma cell lines Daudi and Raji, and Jurkat T cell line were cultured in RPMI-1640 complete medium. Human fibrosarcoma HT1080 cells and U3A cells were cultured in DMEM, HeLa cells in MEME, and human umbilical vein endothelial cells in CS-C medium. All culture media were supplemented with 10% fetal bovine serum (20% in case of Daudi cells) and 100 U/ml of penicillin and 100 μg/ml of streptomycin, and all cells were maintained at 37° C. in 5% CO₂.

Northern Blotting.

Cells were washed twice in phosphate-buffer saline and the total RNA was extracted with Trizol reagent (GIBCO/BRL). RNAs, 20 μg per lane, were separated in 1.2% agarose; 2.2 M formaldehyde gels and transferred to Nylon membranes (Amersham) for 18 to 20 h. RNA was crosslinked to the membrane, incubated in prehybridization solution at 42° C. for 16 h and probed with an EcoRI fragment of PLSCR1 cDNA, glyceraldehyde-3-phosphate dehydrogenase (GAPDH) cDNA or β-actin cDNA labeled with ³²P-dCTP by random priming with the Prime-a-gene labeling system (Promega). Membranes were washed and used to expose x-ray film.

Western Blotting

10⁶ cells were harvested and lysed in 30 μl of cell lysis buffer (2% NP-40 in PBS containing 5 mM EDTA, 50 mM benzamidine, 50 mM N-ethyl maleimide, 1 mM phenylmethylsulfonyl fluoride, and 1 mM leupeptin) at 4° C. for 1 h. Cell lysate was centrifuged at 250,000× g for 30 min at 4° C., and the supernatants denatured (100° C., 5 min) in 10% (w/v) SDS sample buffer containing 2% β-mercaptoethanol. Following SDS-PAGE (0.23 μg total protein per lane) and transfer to nitrocellulose, the membrane was blocked with 4% low fat milk, and incubated for 1 hr at room temperature in presence of 2 μg/ml of 4D2, a monoclonal antibody raised against PLSCR1. The blots were incubated with horseradish peroxidase-conjugated goat anti-mouse IgG (Sigma) for 1 h at room temperature, developed by SuperSignal ULTRA Chemiluminescence (Pierce, Rockford, Ill.), and analyzed on a Kodak Image Station 440CF (Eastman Kodak Co., Rochester, NY). PLSCR1 antigen detected by Western blotting was quantified using Kodak's 1D Image Analysis Software version 3.0.

Protein Concentrations.

Protein concentration of cell lysates was determined by BCA assay. In brief, 150 μl of 1:90 diluted cell lysate were mixed with 150 μl of BCA reagent (Pierce) and incubated at 37° C. for 30 min. Absorbance at 562 nm was measured, and protein concentration calculated using bovine serum albumin as standard.

Confocal Fluorescence Microscopy.

Cells were subcultured on glass cover slips and treated with 1000 IU/ml of IFN-α2a for 0 to 18 hrs at 37° C. All following procedures were performed at room temperature. Cells were washed in PBS, and fixed with 2% paraformaldehyde in PBS for 30 min. After permeabilization by 0.005% saponin in PBS for 5 min, cells were incubated in 2% whole goat serum in PBS for 30 min, followed by incubation with mab 4D2 (20 μg/ml in 2% goat serum in PBS) for 1 hr. Cells were stained with FITC-goat anti-mouse IgG (2μg/ml in PBS) for 1 hr, followed by nuclear counterstain with propidium iodide (0.1 μg/ml in PBS) for 10 min. Cover slips were mounted on glass slides, and samples analyzed on a Bio-Rad MRC 1024 laser scanning confocal microscope attached to a Zeiss Axiovert S100TV microscope with Infinity Corrected Optics (40× oil immersion objective). Images were collected using Bio-Rad's LaserSharp (v3.2) software. Specificity of staining observed for mab 4D2 was evaluated by cell staining with the identical concentration of an isotype-matched antibody raised against complement C9, substituting for mab 4D2.

Molecular Cloning of 5′ Flanking Region of PLSCR1 Gene and Construction of Deletions.

Human PLSCR1 gene was cloned from a BAC-human genomic library (Genome System Inc., St. Louis, Mo.) using full length PLSCR1 cDNA for hybridization, and 4.12 kb of 5′flanking region was sequenced (GenBank AF153715). 4.18 kb DNA consisting of the 5′flanking region (−1 to −4120) and the first 60 bp of the first exon of the gene (+1 to +60) was amplified by PCR using Advantage DNA polymerase mix (CLONTECH Laboratories, Inc., Palo Alto, Calif.), and PCR products were cloned into pGL3-basic-luciferase reporter vector (Promega, Madison. Wis.). Analysis of the 5′ flanking region for the presence of putative binding sites for transcription factors was performed using MatInspector V2.2. The four putative binding sites for ISGF3 or IRFs (see FIG. 5) were deleted by PCR-mediated deletion. All DNA sequencing was performed on an ABI DNA Sequencer Model 373 Stretch (Applied Biosystems) using PRISM Ready Reaction DyeDeoxy Terminator Cycle Sequencing Kit (Perkin Elmer, Foster City, Calif.).

Transfection of Daudi Cells.

Daudi cells were harvested in exponential-growth phase, washed twice and suspended to 1.35×10⁷/ml in OPTI-MEM. To 800 μl of cell suspension in a 0.4 cm electroporation cuvette, 20 μg of pGL3-5′flanking region (or deletions) of PLSCR1 and 20 μg of pSV-β-galactosidase (Promega) were added, and the mixture was incubated for 10 min on ice. Electroporation was performed at 380 V and 500 μF using a Bio-Rad Gene Pulser II (Bio-Rad Laboratories, Hercules, Calif.). Following incubation for 10 min at 37° C., the cells were plated in 10 ml of RPMI-1640 complete medium onto tissue culture plates, and cultured for 24 hrs. Cells were then cultured for an additional 18 hrs in presence or absence of 1000 IU/ml of IFN-α2a, and harvested for luciferase and β-galactosidase assay.

Luciferase and β-galactosidase assay. Luciferase activity was measured using a Luciferase Assay Kit (Promega). In brief, Daudi cells were harvested, washed with PBS, and lysed for 15 min with Reporter lysis buffer. Cell lysates were vortexed for 15 sec and centrifuged at 12,000× g for 2 min at 4° C. In a 96-well plate, 20 μl aliquots of lysate (18 μg of protein) were mixed with 100 μl of luciferase assay buffer by automated injection using a MicroLumatPlus microplate luminometer (EG&G Berthold, Gaithersburg, Md.), and luminescence was measured for a period of 30 sec. β-Galactosidase activity was determined with o-nitrophenyl-β-D-galactopyranoside (OPNG) as a substrate. A 100 μl aliquot of cell lysate (90 μg of protein) was incubated with 100 μl of 4.4 mM ONPG for lh at 37° C., and absorbance read at 420 nm. Luciferase activity was expressed in arbitrary light units, and corrected for transfection efficiency of β-galactosidase.

Evaluation of Cell Surface Exposed PS in Adherent Cells.

The cell surface exposure of PS resulting from treatment with IFN induction and ionophore treatments of adherent cell lines HT1080 and human umbilical vein endothelial cells was evaluated by expression of membrane catalytic function in the prothrombinase enzyme reaction. Cells were grown to about 80% confluence in a 48 well culture plate and induced overnight (18 hr) with either 0 or 1,000 IU/ml IFN-α2a. After three washes, cells were incubated at 37° C. in presence of either 5 μM (HT1080) or 10 μM (HUVEC) A23187 in HBSS containing 2 mM Ca²³⁰ , 0.8 mM Mg²⁺, and 0.1% BSA for the time periods indicated. Controls omitting A23187 received identical 1% (final volume) solvent DMSO. During the last two minutes of treatment with A23187, the prothrombinase reaction was initiated by addition of factor Va (2 nM), factor Xa (1 nM), and prothrombin (1.4 μM). Thrombin generation was terminated by dilution of cell supernatants into HBSS containing 0.1% BSA and 20 mM EDTA, and samples were stored on ice. Aliquots were transferred to a 96-well plate, and thrombin generated was assayed in HBSS containing 0.1% BSA in presence of 150 μM chromogenic substrate CBS 34.47 by monitoring time-dependent changes in absorbance at 405 nm using a Thermoₘₐₓ plate reader (Molecular Devices, Sunnyvale, Calif.).

Flow Cytometry.

IFN and A23187-induced cell surface exposure of PS was evaluated in the suspension cell lines Daudi and Raji using flow cytometric detection of bound factor Va (light chain) as previously described. Following 18 hr induction with either 0 or 1,000 IU/ml IFN-α2a , cells were washed once with RPMI and suspended (3×10⁶ cells/ml) in RPMI containing 0.1% BSA, 4 mM Ca²⁺. After 2 min at 37° C., A23187 (0 or 1 μM) was added. At each time point, reaction was stopped by addition of 10 mM EGTA, and cells incubated with bovine factor Va (10 μg/ml; 15 min at room temperature) and bound factor Va was detected with mab FITC-V237 specific for the light chain. Cells staining positive for bound factor Va were analyzed by flow cytometry (FACSCalibur, Becton Dickinson). Data were expressed as percentage of gated factor Va-positive cells in total cell population.

### EXAMPLE 16

Previous screening by high density oligonucleotide microarrays provided evidence of an induction of PLSCR1 mRNA in HT1080 cells 6 hr after exposure to IFN-α, β or γ. These findings were extended by demonstrating the time-dependent induction of both PLSCR1 mRNA and protein by IFN-α2a in Northern and Western blots. Increased PLSCR1 mRNA was detected by 3 hr after IFN-α2a (1,000 IU/ml) addition, with protein expression increasing to approximately 10-fold above basal levels at 18 hrs. Peak expression of PLSCR1 mRNA was observed at 6 hrs, the same length of IFN treatment as in the microarray analysis. By contrast to this response observed in the IFN-responsive HT1080 cells, treatment with IFN-α2a had no affect on PLSCR1 expression in mutant U3A cells, an HT1080 derivative cell line deficient in Stat1 transcription factor required for signaling through IFN-receptors.

The IFN-α2a -dependence of PLSCR1 expression observed in HT1080 cells was confirmed in a variety of other transformed cell lines as well as in cultures of human umbilical vein endothelial cells, and, non-transformed peripheral blood mononuclear cells isolated from whole blood. In all cases, incubation with IFN-α2a caused a marked increase in PLSCR1 protein expression, ranging to as high as 10-fold above basal levels in the Raji and Daudi cell lines. These data indicate that the PLSCR1 gene is highly upregulated by IFN-α2a treatment in a variety of normal and transformed cell types.

After IFN treatment, newly synthesized PLSCR1 was detected in the plasma membrane where it appeared to concentrate in membrane protrusions. In addition to plasma membrane, PLSCR1 antigen also appeared to be distributed in a variety of other intracellular membranous structures, suggestive of golgi and endoplasmic reticulum.

Inspection of PLSCR1 genomic sequence revealed three potential IFN regulated sites within the first 4 kbp of 5′-flanking sequence; a potential binding site for IRF-2 at (−3815)gaaaagaGAATcc(−3800); potential binding sites for ISGF3 at (−2733)acaaaaaGAAAgc(−2721) and at (−2519)aaaaacaGAAAcc(−2497), and a single consensus ISRE in the untranslated exon 1 at (+21)ggaaaagGAAAcc(+35) (FIG. 14). In order to identify which of these four putative regulatory sites actually contributed to the observed IFN-inducible expression PLSCR1, luciferase reporter constructs incorporating 5′ untranslated PLSCR1 gene sequence spanning one or more of the putative sites were expressed in Daudi cells, and the response of the transfected cells to IFN-α2a was determined. As shown in FIG. 14, these experiments revealed that the IFN-inducible expression of PLSCR1 appears to be controlled by the single ISRE that is located in exon 1. The close proximity of this ISRE to the PLSCR1 transcriptional start site may account for the observed potency of IFN-α2a in inducing PLSCR1 expression.

In reconstituted proteoliposomes, PLSCR1 has been shown to mediate accelerated transbilayer migration of membrane phospholipids in presence of Ca²⁺ or under acidic conditions. Furthermore, the level of expression of this protein was generally found to correlate with the extent to which phosphatidylserine was exposed at the cell surface following calcium ionophore treatment, suggesting that PLSCR1 participates in the remodeling of plasma membrane phospholipids in activated platelets and injured or apoptotic cells exposed to increased intracellular [Ca²⁺]. A potential influence of PLSCR1 on either cell proliferation or cell clearance in vivo was also suggested by the observation of altered transcription—including alternative splicing- of a murine PLSCR1 orthologue in leukemogenic versus non-leukemogenic cell clones. We therefore considered whether the marked upregulation of PLSCR1 induced by IFN-α2a is also accompanied by changes in the plasma membrane that might increase the likelihood of phosphatidylserine becoming exposed at the cell surface. Despite the presumed activity of PLSCR1 in mediating accelerated transbilayer movement of plasma membrane phospholipids leading to transfer of phosphatidylserine to the outer leaflet, we were not able to detect any change in the IFN-α2a-treated cells indicative of increased surface exposure of plasma membrane phosphatidylserine.

These experiments demonstrate that the PLSCR1 gene is a member of the IFN-stimulated gene family requiring JAK/STAT signaling for optimal expression. The locus of the controlling ISRE in the untranslated first exon of the PLSCR1 gene represents a putative binding site for the ISGF3 transcription factor complex. Whereas most known ISREs map to flanking sequence that is 5′ to the transcriptional start site, the location of an active ISRE in untranslated exonic sequence has also previously been described for the p202 gene, another interferon-stimulated gene regulated through ISGF3. As has been noted in other interferon-stimulated genes, the marked effect of IFN-α2a in upregulating PLSCR1 expression is consistent with the relatively close proximity of this single active ISRE to the transcriptional start site.

Certain of the interferon stimulated genes regulated through ISREs are thought to be involved in the apoptotic, antiproliferative, and tumor suppressive activities of IFN-α, although the precise roles of the downstream effector genes actually responsible for these activities remain to be resolved. Induction of apoptosis of malignant cells or virus-infected cells by interferons, and clearance of these apoptotic cells by the reticuloendothelial system, are widely assumed to underlie the therapeutic response to interferon treatment. In light of the putative role of the PLSCR1 gene product in catalyzing movement of phospholipids between plasma membrane leaflets, it is of particular interest that one of the most prominent changes observed in apoptotic cells is a remodeling of the topology of plasma membrane phospholipids, with surface exposure of PS and other aminophospholipids that are normally sequestered to the inner leaflet. Such cell surface exposure of PS has been implicated in promoting clearance of injured or apoptotic cells by the reticuloendothelial system.

Phospholipid scramblase is an endofacial-oriented plasma membrane protein that has been proposed to contribute to accelerated movement of phospholipids between plasma membrane leaflets in activated platelets as well as in injured and apoptotic cells that are exposed to local elevations in [Ca²⁺] or to acidification affecting the inner plasma membrane leaflet. This activity of the PLSCR1 gene product in promoting Ca²⁺ and pH-dependent movement of phospholipids between membrane leaflets was demonstrated in reconstituted proteoliposomes containing this protein, and the level of cellular expression of PLSCR1 was previously found in general to correlate with the observed extent of transfer of PS to the cell surface in response to induced elevations of cytoplasmic [Ca²⁺]. Nevertheless, the exact role of this protein in promoting transbilayer movement of PS and other plasma membrane phospholipids, and the actual mechanism of activation of the phospholipid scramblase pathway in situ, remains to be clarified. As noted above, induction of PLSCR1 by IFN-α2a leads to a marked increase in concentration of phospholipid scramblase that is expressed in the plasma membrane of the IFN-treated cells, but we were unsuccessful in detecting either a corresponding increase in surface-exposed PS or increased sensitivity of the plasma membranes of these cells to subsequent treatment with calcium ionophore. These data suggest that the mobilization of PS to the cell surface cannot simply be attributed to the level of expression of the PLSCR1 gene product as was previously assumed, but is likely to require additional factors, including potentially another protein, that either acts directly on the plasma membrane or that interacts with PLSCR1 to accelerate transbilayer movement of phospholipids in the plasma membrane. Alternatively, the endogenous level of PLSCR1 expressed in the plasma membrane of these cells before IFN treatment may itself be sufficient to mediate maximal response to calcium ionophore under the conditions of these experiments, masking more subtle changes in phospholipid trafficking between plasma membrane leaflets arising from the IFN-induced increase in plasma membrane concentration of the protein. It also remains to be determined whether the marked increase in PLSCR1 expression induced by IFN promotes remodeling of plasma membrane phospholipids or cell clearance in vivo, under physiological conditions relevant to tumor growth and viral infection.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

PHOSPHOLIPID SCRAMBLASES (PLSCR)

| SEQ ID NO | NAME | TYPE |
|---|---|---|
| 1 | Human PLSCR1 | nucleotide |
| 2 | | polypeptide |
| 3 | Human PLSCR2 | nucleotide |
| 4 | | polypeptide |
| 5 | Human PLSCR3 | nucleotide |
| 6 | | polypeptide |
| 7 | Human PLSCR4 | nucleotide |
| 8 | | polypeptide |
| 9 | Mouse PLSCR1 | nucleotide |
| 10 | | polypeptide |
| 11 | Mouse PLSCR2 | nucleotide |
| 12 | | polypeptide |
| 13 | Mouse PLSCR3 | nucleotide |
| 14 | | polypeptide |
| 15 | Mouse PLSCR4 | nucleotide |
| 16 | | polypeptide |

What is claimed is:

1. An isolated polynucleotide selected from the group consisting of:
(a) SEQ ID NO:3, SEQ ID NO:5, SEQ ID NO:7, SEQ ID NO:9, SEQ ID NO:13 or SEQ ID NO:15;
(b) the sequence of (a), wherein T can also be U;
(c) nucleic acid sequences complementary to the sequence of a); and
(d) fragments of (a), (b), or (c) that are at least 15 bases in length and that will hybridize under moderate to highly stringent conditions to DNA that encodes the Phospholipid Scramblase protein of SEQ ID NO:4, SEQ ID NO:6, SEQ ID NO:8, SEQ ID NO:1O, SEQ ID NO:14 or SEQ ID NO: 16, respectively.

2. An expression vector containing a polynucleotide of claim 1.

3. The vector of claim 2, wherein the vector is a plasmid.

4. The vector of claim 2, wherein the vector is a viral vector.

5. A host cell containing a vector of claim 2.

6. The host cell of claim 5, wherein the cell is prokaryotic.

7. The host cell of claim 5, wherein the cell is eukaryotic.

8. A substantially purified Phospholipid Scramblase polypeptide, wherein the polypeptide is encoded by polynucleotide as set forth in SEQ ID NO:3, SEQ ID NO:5, SEQ ID NO:7, SEQ ID NO:13 or SEQ ID NO:15.

9. A substantially purified Phospholipid Scramblase polypeptide, wherein the polypeptide has an amino acid sequence as set forth in SEQ ID NO:4, SEQ ID NO:6, SEQ ID NO:8, SEQ ID NO: 14 or SEQ ID NO: 16, or fragments thereof.

10. Antibodies which bind to a polypeptide of claim 8, or fragments thereof.

11. The antibodies of claim 10, wherein the antibodies are polyclonal.

12. The antibodies of claim 10, wherein the antibodies are monoclonal.

13. A method for producing a polypeptide comprising an amino acid sequence of SEQ ID NO:4, SEQ ID NO:6, SEQ ID NO:8, SEQ ID NO:10, SEQ ID NO:14 or SEQ ID NO:16, the method comprising:
(a) culturing a host cell of claim 5 under conditions suitable for the expression of the polypeptide; and
(b) recovering the polypeptide from the host cell culture.

14. An isolated nucleic acid sequence comprising a non-coding regulatory sequence isolated upstream from a Phospholipid Scramblase gene, wherein the nucleic acid sequence contains at least one restriction site for cloning a heterologous nucleic acid sequence of interest.

15. The isolated nucleic acid sequence of claim 14, wherein the sequence is operably linked to a heterologous nucleic acid sequence thereby forming a DNA construct.

16. The isolated nucleic acid sequence of claim 14 wherein the heterologous nucleic acid sequence is selected from the group consisting of a nucleic acid sequence encoding a selectable marker gene, the glucuronidase (GUS) reporter gene, and the luciferase (LUC) reporter gene.

17. A method of identifying a compound that modulates expression of a Phospholipid Scramblase polypeptide, wherein the method comprises;
(a) incubating the compound with a cell containing the DNA construct of claim 15 under conditions sufficient to permit the compound to interact with the construct;
(b) detecting expression of the heterologous gene in the presence of the compound wherein an increase or decrease in the expression of the heterologous gene in the presence of the compound compared to expression in the absence of the compound identifies compounds that modulate Phospholipid Scramblase polypeptide expression.

18. The method of claim 17, wherein the modulation is inhibition of Phospholipid Scramblase expression.

19. The method of claim 17, wherein the modulation is stimulation of Phospholipid Scramblase expression.

20. The method of claim 17, wherein the compound is selected from the group consisting of peptides, peptidomimetics, polypeptides, pharmaceuticals, chemical compounds, biological agents, antibodies and trophic agents.

21. A transgenic knockout mouse whose genome comprises a disruption of a Phospholipid Scramblase polypeptide gene, wherein said disruption comprises the insertion of a transgene comprising a selectable marker sequence, and wherein said disruption results in the mouse exhibiting a higher susceptibility to viral infection or cancer as compared to a wild-type mouse.

22. The transgenic knockout mouse of claim 21, wherein the mouse is homozygous or heterozygous for said disruption of the endogenous Phospholipid Scramblase polypeptide gene.

23. The transgenic knockout mouse of claim 22, wherein the endogenous Phospholipid Scramblase polypeptide gene contains the polynucleotide sequence as set forth in SEQ ID NO:9, SEQ ID NO:11, SEQ ID NO:13 or SEQ ID NO: 15.

24. A method for producing a transgenic mouse exhibiting an increased susceptibility to viral infection or to cancer, said method comprising:
(a) introducing a transgene comprising a selectable marker sequence into a mouse embryonic stem cell;
(b) introducing the mouse embryonic stem cell into a mouse embryo;
(c) transplanting the embryo into a pseudopregnant mouse;
(d) allowing the embryo to develop to term; and
(e) identifying a transgenic mouse whose genome comprises a disruption of the endogenous Phospholipid Scramblase polypeptide gene, wherein the disruption results in the mouse exhibiting an increased susceptibility to viral infection or to cancer as compared to a wild-type mouse.

25. The method of claim 24, wherein the transgenic mouse is homozygous or heterozygous for the disruption of the endogenous Phospholipid Scramblase polypeptide gene.

26. A method of inhibiting or preventing viral infection comprising introducing into viral-infected cells or uninfected cells a Phospholipid Scramblase polypeptide or fragments thereof containing the amino acid sequence PPxY.

27. The method of claim 26, wherein the viral infection is an infection of a virus selected from the group consisting of a rhabdovirus, a filovirus, a retrovirus, a flavivirus, a coronavirus, a orthomyxovirus, a bunyavirus, a hepadnavirus, a herpesvirus, a poxvirus, a togavirus, a iridovirus, a paramyxovirus and a arenavirus.

28. The method of claim 27, wherein the viral infection is selected from the group consisting of an HIV infection, an Ebola virus infection, a Marburg virus infection and a Rabies virus infection.

29. The method of claim 26, wherein the virus infection is an infection of a membrane-bound virus.

30. The method of claim 26, wherein the polypeptides and fragments bind to proteins containing one or more WW domain sequence motifs.

31. The method of claim 26, wherein the Phospholipid Scramblase polypeptide is interferon-inducible.

32. The method of claim 31, wherein the Phospholipid Scramblase polypeptide has the amino acid sequence as set forth in SEQ ID NO:2.

33. The method of claim 26, further comprising administering an interferon.

34. The method of claim 26 wherein the fragments are peptidomimetics.

35. A method for identifying a compound that modulates Phospholipid Scramblase polypeptide activity:
(a) incubating the compound with a cell expressing a Phospholipid Scramblase polypeptide under conditions sufficient to permit the compound to interact with the cell;
(b) comparing the cellular response in a cell incubated with the compound with the cellular response of a cell not incubated with the compound, thereby identifying a compound that modulates Phospholipid Scramblase polypeptide activity.

36. The method of claim 35, wherein the cellular response is a decrease in Phospholipid Scramblase polypeptide activity.

37. The method of claim 35, wherein the cellular response is an increase in Phospholipid Scramblase polypeptide activity.

38. A method of treating a disorder associated with Phospholipid Scramblase polypeptide activity comprising administering to a subject in need thereof a therapeutically effective amount of a compound that modulates a Phospholipid Scramblase polypeptide activity.

39. The method of claim 38, wherein the disorder is a viral infection or a cancer.

40. The method of claim 3 8, wherein the viral infection is an infection of a rhabdovirus, a filovirus, a retrovirus, a flavivirus, a coronavirus, a orthomyxovirus, a bunyavirus, a hepadnavirus, a herpesvirus, a poxvirus, a togavirus, a iridovirus, a paramyxovirus or a arenavirus, an infection of a rhabdovirus, a filovirus or a retrovirus.

41. The method of claim 40, wherein the viral infection is an HIV infection, an Ebola virus infection, a Marburg virus infection or a Rabies virus infection.

42. The method of claim 39, wherein the cancer is hairy cell leukemia, chronic myelogenous leukemia, myeloma, melanoma, renal cell carcinoma, Kaposi's sarcoma, follicular lymphoma, thrombocythemia or erythroleukemia.

43. The method of claim 38, wherein the compound comprises an agonist or antagonist of a Phospholipid Scramblase polypeptide activity.

44. The method of claim 38, wherein the compound is selected from the group consisting of peptides, peptidomimetics, polypeptides, pharmaceuticals, chemical compounds, biological agents, antibodies and trophic agents.

45. The method of claim 38, wherein the Phospholipid Scramblase polypeptide activity is activity of a polypeptide as set forth in SEQ ID NO:4, SEQ ID NO:6 or SEQ ID NO:8.

46. The method of claim 45, wherein the Phospholipid Scramblase polypeptide is interferon-inducible.

47. The method of claim 46, wherein the Phospholipid Scramblase polypeptide has the amino acid sequence as set forth in SEQ ID NO:2.

48. The method of claim 38, further comprising administering an interferon.

49. A method of diagnosis of a subject having or at risk of having a Phospholipid Scramblase-related disorder comprising detecting in the subject a level or activity of a Phospholipid Scramblase polypeptide wherein a difference in the level or activity of a Phospholipid Scramblase polypeptide in the subject from a level or activity of a Phospholipid Scramblase polypeptide in a normal subject is indicative of a Phospholipid Scramblase-related disorder.

50. The method of claim 49, wherein the Phospholipid Scramblase-related disorder is cancer or virus infection.

51. The method of claim 50, wherein the cancer is hairy cell leukemia, chronic myelogenous leukemia, myeloma, melanoma, renal cell carcinoma, Kaposi's sarcoma, follicular lymphoma, thrombocythemia or erythroleukemia.

52. The method of claim 50, wherein the virus infection is an infection of a rhabdovirus, a filovirus, a retrovirus, a flavivirus, a coronavirus, a orthomyxovirus, a bunyavirus, a hepadnavirus, a herpesvirus, a poxvirus, a togavirus, a iridovirus, a paramyxovirus or a arenavirus, an infection of a rhabdovirus, a filovirus or a retrovirus.

53. The method of claim 52, wherein the virus infection is an HIV infection, an Ebola virus infection, a Marburg virus infection or a Rabies virus infection.

54. The method according to claim 49, wherein the level or activity of a Phospholipid Scramblase polypeptide in the subject having or at risk of having Phospholipid Scramblase-related disorder is lower than the level of a of Phospholipid Scramblase polypeptide in a normal subject.

55. A method of increasing or extending the viability of mammalian cells or tissues by inhibiting the expression of a Phospholipid Scramblase polynucleotide within the cell or tissue.

56. The method according to claim 55, wherein the polynucleotide is selected from:
(a) SEQ ID NO:3, SEQ ID NO:5, SEQ ID NO:7, SEQ ID NO:9, SEQ ID NO:11, SEQ ID NO:13 or SEQ ID NO:15
(b) the sequence of (a), wherein T can also be U;
(c) nucleic acid sequences complementary to the sequence of (a); and
(d) fragments of (a), (b), or (c) that are at least 15 bases in length and that will hybridize under moderate to highly stringent conditions to DNA that encodes the Phospholipid Scramblase protein of SEQ ID NO:4, SEQ ID NO:6, SEQ ID NO:8, SEQ ID NO: 10, SEQ ID NO:12, SEQ ID NO:14 or SEQ ID NO:16, respectively.

57. The method according to claim 55, wherein the polynucleotide is selected from the group consisting of SEQ ID NO: 1; SEQ ID NO: 1, where T can also be U; nucleic acid sequences complementary thereto; and fragments thereof that are at least 15 bases in length and that will hybridize under moderate to highly stringent conditions to DNA that encodes the Phospholipid Scramblase protein of SEQ ID NO:2.

58. A method of treating a subject having or at risk of having a disorder associated with a Phospholipid Scramblase polypeptide or polynucleotide comprising:
introducing into a subject having or at risk of having the disorder a polynucleotide encoding the Phospholipid Scramblase polypeptide operatively linked to a regulatory sequence, thereby treating the subject.
